(19) United States
(12) Patent Application Publication
(10) Pub. No.: US 2021/0346420 A1
(43) Pub. Date: Nov. 11, 2021

(54) COMBINATION IMMUNOTHERAPIES

(71) Applicant: The Regents of the University of California, Oakland, CA (US)

(72) Inventor: Gen-Sheng Feng, La Jolla, CA (US)

(21) Appl. No.: 17/283,683

(22) PCT Filed: Oct. 10, 2019
(86) PCT No.: PCT/US2019/055613

§ 371 Date: Apr. 8, 2021

Related U.S. Application Data
(60) Provisional application No. 62/744,035, filed on Oct. 10, 2018.

(51) Int. Cl. A61K 31/713; A61K 39/395; A61P 35/00; C07K 16/28

(52) U.S. Cl. CPC A61K 31/713; A61K 39/3955; A61K 2039/505; C07K 16/2827; A61P 35/00

(57) ABSTRACT

Provided herein are combination immunotherapies and methods of use for cancer treatment, in particular for the treatment of early and late stage liver cancer. Specifically, administration of polyIC with a PD-L1 antibody together effectively suppresses tumor progression after oncogene delivery.

### CROSS-REFERENCE TO RELATED APPLICATIONS

This application claims priority to U.S. Provisional Application Ser. No. 62/744,035, filed on Oct. 10, 2018, which is herein incorporated by reference in its entirety.

### FEDERALLY SPONSORED RESEARCH OR DEVELOPMENT

This invention was made with government support under Grant No. R01CA176012 and Grant No. R01CA188506 awarded by the National Institutes of Health. The government has certain rights in the invention.

### BACKGROUND

Primary liver cancer, with the majority being hepatocellular carcinoma (HCC), is now the second leading cause of cancer mortality and the fifth most common cancer worldwide, claiming approximately 800,000 lives every year. HCC is a chemotherapy-resistant tumor with limited treatment options, including, surgical resection, liver transplantation, and local ablation at the early stages. Sorafenib, a multi-kinase inhibitor, remains a first-line systemic drug for advanced HCC even with poor outcomes, and similar low therapeutic benefits were reported for regorafenib, lenvatinib, and cabozantinib. Over 100 clinical trials that tested other compounds or approaches have failed to show therapeutic benefit to HCC patients.

Immunotherapy by blocking inhibitory pathways in T lymphocytes, such as the PD-L1/PD-1 axis, is being widely tested in various solid tumors. Notably, this emerging therapeutic approach is already in clinical trials for advanced HCC in multi-centers around the globe. Two latest reports on open-label, non-randomized, phase 1/2 trials with pembrolizumab or nivolumab indicated manageable safety in advanced HCC patients with or without prior sorafenib treatment, albeit with very limited therapeutic benefits observed so far. The outcome of immunotherapy for liver cancer can be compounded by the unique immunotolerant microenvironment in the liver. A variety of clinical trials are ongoing to evaluate combination of immune checkpoint inhibitors or with other drugs, without clear justification or support by preclinical data.

Unexpectedly, a synthetic double stranded RNA (dsRNA), polyinosinic-polycytidylic acid (polyIC), was previously identified to have a potent liver tumor-inhibitory role. Injection of polyIC at the pre-cancer stage effectively prevented liver tumor initiation in several mouse models. However, injection of polyIC at the post-cancer stage showed no inhibition on tumor progression.

### SUMMARY

Provided herein are combination immunotherapies and methods of use in cancer treatment, in particular for the treatment of early and late stage liver cancer.

In one aspect, a method of treating a cancer in a subject in need thereof, the method comprising administering a therapeutically effective amount of a double stranded RNA (dsRNA).

In one aspect, a method of treating a cancer in a subject in need thereof, the method comprising administering a therapeutically effective amount of a double stranded RNA (dsRNA) and an antibody.

In some embodiments, the dsRNA is a TLR3 ligand.

In some embodiments, the dsRNA is a TLR3 agonist.

In some embodiments, the dsRNA is selected from the group consisting of polyadenylic-polyuridylic acid (polyAU) or polyinosinic-polycytidylic acid (polyIC, polyrl, polyrC).

In some embodiments, the dsRNA is polyIC.

In some embodiments, the dsRNA inhibits cancer tumor initiation.

In some embodiments, the dsRNA inhibits liver cancer tumor initiation.

In some embodiments, the administration of the dsRNA results in an increased number of natural killer (NK) cells.

In some embodiments, the administration of the dsRNA results in an increased number of macrophages.

In some embodiments, the administration of the dsRNA results in increased expression of an immune system checkpoint component.

In some embodiments, the administration of the dsRNA results in increased expression of a programmed cell death receptor ligand.

In some embodiments, the administration of the dsRNA results in increased expression of a programmed cell death-1 receptor (PD-1) ligand.

In some embodiments, the administration of the dsRNA results in increased expression of one or more of the following PD-1 ligands: a programmed cell death ligand-1 (PD-L1) and programmed cell death ligand-2 (PD-L2).

In some embodiments, the administration of the dsRNA results in increased expression of PD-L1.

In some embodiments, the antibody is against an immune system checkpoint component.

In some embodiments, the antibody is an anti-PD-L1 antibody.

In some embodiments, the cancer is a liver cancer.

In some embodiments, the cancer is primary liver cancer.

In some embodiments, the cancer is late stage liver cancer.

In some embodiments, the cancer is metastatic colon cancer with liver tumors.

In some embodiments, the cancer is hepatocellular carcinoma (HCC).

In some embodiments, the cancer is initiated by an oncogene selected from the group consisting of N-Ras, c-Myc, c-Met, or a truncated beta-catenin mutant.

In some embodiments, the cancer is initiated by N-Ras and c-Myc oncogenes.

In some embodiments, the cancer in initiated by c-Met and truncated beta-catenin mutant oncogenes.

In some embodiments, the administration of dsRNA and antibody results in an increased number of cytotoxic T cells.

In some embodiments, the administration of dsRNA and antibody results in an increased number of CD8+ cytotoxic T cells.

In some embodiments, the administration of dsRNA and antibody results in a sustained increased number of CD8+ cytotoxic T cells.

In some embodiments, the administration of dsRNA and antibody results in an increased number of CD45+ cells.

In some embodiments, the administration of the dsRNA and antibody suppresses cancer progression.

In some embodiments, the administration of the dsRNA and antibody suppresses liver cancer progression.

In some embodiments, the administration of the dsRNA and antibody results in decreased tumor burdens.

In some embodiments, the administration of the dsRNA and antibody results in activation of one or more of the following immune system responses: innate and adaptive.

In some embodiments, the method is able to guide the design of successful clinical trials for liver cancer.

In one aspect, a pharmaceutical combination comprising a dsRNA and an antibody.

In some embodiments, the dsRNA is polyIC.

In some embodiments, the antibody is anti-PD-L1.

In some embodiments, the dsRNA and antibody are administered as a fixed combination.

In some embodiments, the dsRNA and antibody are administered as a non-fixed combination.

In some embodiments, the dsRNA and antibody are administered sequentially.

In some embodiments, the dsRNA and antibody are administered concurrently.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows exemplary polyIC inhibits liver tumor initiation but not progression. (A) The scheme of experimental procedure for polyIC treatment. Mice were i.p. injected of polyIC (4 μg/g) at −10, −8, −6, −4 and −2 days before (Pre-polyIC), or 14, 16, 18, 20 and 22 days after N-Ras/c-Myc (Ras/Myc) transfection via HTVi (Post-polyIC), and mice were sacrificed (SAC) at 4 or 6 weeks (4 w or 6 w) after oncogene injection, for phenotypic analysis. (B) Representative macroscopic views and H&E staining of liver sections of WT control, pre-polyIC and post-polyIC treatments. Magnification: ×20; Scale bar: 50 m. (C-E) Tumor burdens were calculated by (C) liver weight/body weight (LW/BW) ratios, (D) maximal diameters (mm) or numbers (E) of tumor nodules. Data in (C-E) are presented as means±SD (n=4-5, *p<0.05, **p<0.01) for any other groups versus the WT control group.

FIG. 2 shows exemplary roles of innate immunity cells in mediating polyIC inhibition of tumor initiation. (A) The scheme of experimental procedure. Mice were divided into four groups. In the first two groups (WT+PBS; WT+polyIC), WT mice were injected i.p. with PBS or polyIC (4 μg/g of body weight), at −10, −8, −6, −4, −2 days before sacrifice (SAC) for analysis. The other two groups (GFP+polyIC; Ras/Myc+polyIC) of mice were injected with GFP or N-Ras/c-Myc plasmids at day 0, and were then i.p. injected of polyIC (4 μg/g) at day 14, 16, 18, 20 and 22, before sacrifice (SAC) at day 24. The representative H&E stained liver sections are shown for each group. Magnification: ×20; Scale bar: 50 m. (B) Flow cytometry analysis was performed and the relative cell numbers of innate and adaptive immune cell subsets were quantified in the livers of the four groups. (C) Tumor burdens were calculated by LW/BW ratios, maximal diameters (mm) and numbers of tumor nodules, to evaluate the effects of depleting NK cells (NK1.1 Ab), macrophages (clondronate liposome, C.L.) or CD8 T cells (CD8 Ab) on polyIC inhibition of HCC initiation. Data in (B-C) are represented as means±SD (n=5-7, *p<0.05, **p<0.01) for any other group versus WT group, or as indicated by the horizontal lines.

FIG. 3 shows exemplary polyIC upregulates PD-L1 expression in LSECs. (A) Immunoblot analysis of PD-L1 expression in liver lysates of four groups, as in FIG. 2A with GAPDH as loading control. (B) Immunostaining of PD-L1 (green) and VE-cadherin (red) in liver sections, magnification: ×40; scale bar: 25 m. (C) Flow cytometry analysis to show the representative PD-L1 expression in LSECs and other NPCs (non-LSECs) in livers as indicated. (D) Quantification of mean fluorescence intensity (MFI) of PD-L1 expression in LSECs and non-LSECs in four groups of livers. (E) Relative mRNA levels of PD-L1 expression of PD-L1 in isolated LSECs after treatment with PBS or polyIC (80 μg/mL) for 2 days in vitro. (F) Flow cytometry analysis and quantification for the ratios of PD-1+ cells in CD4+, CD8+ and B lymphocytes in livers as indicated. Data in (D, F) are represented as means SD (n=6) for any other groups versus WT+PBS, or indicated by a horizontal line. Data in (E) are represented as means±SD (n=4). *p<0.05, **p<0.01.

FIG. 4 shows exemplary polyIC sensitizes PD-L1 blockade in HCC therapy in mice. (A) The scheme of experimental procedure for polyIC, PD-L1 Ab or their combination treatment (Combo). N-Ras/c-Myc were transfected into all four groups of mice at day 0. polyIC (4 μg/g) (or PBS) was i.p. injected at day 14, 16, 18, 20, and 22, and PD-L1 Ab (or isotype IgG) was i.p. injected at day 17, 19, and 21. All mice were sacrificed (SAC) 6 weeks after oncogene transfection. (B) Representative macroscopic views and H&E stained liver sections in mice of control, polyIC, anti-PD-L1 and Combo treatment. Magnification: ×20; Scale bar: 50 μm. (C) Tumor loads were calculated by LW/BW ratios, maximal diameters (mm) and numbers of nodules, with the ratios of spleen weight/body weight (SW/BW) also measured. (D) Left: representative immunostaining of Ki67 in liver tumor areas in liver sections. Magnification: ×40; Scale bar: 25 μm. Right: quantification of Ki67+ tumor cell numbers per field. (E) Kaplan-Meier survival curves of overall survival of the four groups of mice (n=8-9). Log-rank test was performed. (F) Tumor burdens were measured at 6 weeks after Ras/Myc transfection and the combination treatment starting at 3 or 4 weeks after oncogene transfection, Combo (3 w) and Combo (4 w), the control mice were transfected with Ras/Myc without receiving the treatment. Data in (C, D, F) are represented as means±SD (n=7-9, n=5, n=6-10, respectively). *p<0.05, **p<0.01, for any other group versus control group, or as indicated by horizontal line in (E).

FIG. 5 shows exemplary combined treatment of polyIC and PD-L1 Ab boosts innate and adaptive immunity in the liver. (A) N-Ras/c-Myc were transfected into the mice at day 0. polyIC (4 μg/g) (or PBS) was i.p. injected at day 14, 16, 18, 20, and 22 and PD-L1 Ab (or isotype IgG) was i.p. injected at day 17, 19, and 21. All mice were sacrificed (SAC) at day 24 for analysis. Top: representative immunostaining of CD45 in liver sections. Magnification: ×40; Scale bar: 25 μm. Bottom: quantification of CD45+ cell numbers isolated by MACS per mouse, with the WT group indicating mice without any treatment. (B) Flow cytometry analysis was performed and quantified for the relative ratios of macrophages, NK, DC and MDSC in the total NPCs. (C) Flow cytometry analysis was performed and quantified for the relative ratios of CD4, CD8 cells and B cells in the total NPCs, and regulatory T cell (Treg) in the CD4+ cells. Data are represented as mean±SD (A: n=3; B-C: n=6). *p<0.05, **p<0.01, for any other group versus control group, or as indicated by horizontal line in (B, C).

FIG. 6 shows exemplary enhanced anti-tumor effect of polyIC and PD-L1 blockade is cytotoxic T cell dependent. (A) Flow cytometry analysis was performed to determine the ratios of cytotoxic T cell proliferation (Ki67+), activation (CD44+CD62L), and cytotoxic function (granzyme B+), in livers of four groups, as shown in FIG. 5A. (B) Immunostaining of CD8 in tumor and non-tumor areas in liver sections, as shown in FIG. 4A. Magnification: ×40; Scale bar: 25 μm. (C) Quantification of CD8+ cell numbers per field in the tumor and non-tumor areas, respectively, in the liver sections, related to panel B. (D) The scheme of experimental procedure for CD8 blockade. Ras/Myc were transfected into all mice at day 0. For the combination treatment, mice received polyIC (4 μg/g) at day 14, 16, 18, 20, 22, and PD-L1 Ab (200 μg) at day 17, 19, and 21. For CD8 blockade, CD8 Ab (200 μg) (or isotype IgG, 200 μg) was i.p. injected at day 13, 18, 23, 28, and 33. All mice were sacrificed (SAC) 6 weeks after oncogene transfection. (E) Tumor burdens were evaluated as indicated, related to panel D. Data in (A, C, E) are represented as means SD (n=5-7). *p<0.05, **p<0.01, for any other group versus control group, or indicated by horizontal line.

FIG. 7 shows an exemplary model for the tumor-suppressing effects of polyIC and/or PD-L1 blockade. Injection of the synthetic dsRNA polyIC suppresses tumor initiation by activation of multiple innate immune cell functions, and its induction of PD-L1 expression in LSECs sensitizes liver response to anti-PD-L1 blockade. Thus, a combined treatment of polyIC and PD-L1 Ab may be an effective combination immunotherapy for liver cancer. NK: Natural killer cell; DC: Dentritic cell; LSEC: liver sinusoidal endothelial cell; effCD4 cell: Effective CD4 T cell; Treg: regulatory T cell; exhCD8 T cell: exhausted CD8 T cell; aCTL: activated cytotoxic T cell.

FIG. 8 shows exemplary liver tumors were inducted by hydrodynamic tail vein injection (HTVi) of oncogenes N-Ras and c-Myc (Ras/Mcy) in mice. (A) The scheme of experimental procedure. Two expression constructs for human N-Ras (0.95 μg/g) and human c-Myc (0.05 μg/g), together with a plasmid expressing Sleeping Beauty transposase (SB) (0.1 μg/g) were co-transfected, and mice were sacrificed (SAC) for analysis at 2, 4, and 6 weeks. (B) Representative macroscopic views and H&E staining of liver sections are shown for WT and oncogene-transfected mice. Magnification: ×20; Scale bar: 50 μm. (C-E) Tumor burdens were determined by (C) liver weight/body weight (LW/BW) ratios, (D) maximal diameters (mm), and (E) numbers of tumor nodules. (F) Spleen weight/body weight (SW/BW) ratios. Data in (C-F) are represented as means±SD (n=3-4). *p<0.05, **p<0.01, for any other groups versus WT control.

FIG. 9 shows exemplary polyIC treatment does not affect genomic integration of exogenous DNA in hepatocytes. (A) The scheme of experimental procedure. Mice were treated by i.p. injection of polyIC (4 μg/g) (or PBS) at −10, −8, −6, −4, −2 days, before transfection via HTVi of plasmids expressing GFP (1 μg/g) and Sleeping Beauty (0.1 μg/g). Genomic DNAs were extracted from mouse livers for PCR analysis at 7 days after HTVi. (B) Quantitative real time-PCR detected GFP cDNA integrated into the genomic DNA in the livers of the two groups. Data are represented as means±SD (n=3).

FIG. 10 shows exemplary depletion efficiency of macrophages, NK and CD8+ cells. (A) The scheme of experimental procedure for cell depletion. Mice were i.p. injected of polyIC (4 μg/g) (or PBS), in combination with NK1.1 Ab (600 μg/g), clondronate liposome (200 μl), or CD8 Ab (200 μg) as shown. (B) Flow cytometry analysis was performed to show the relative numbers of macrophages, NK and CD8+ cells in each group, with or without polyIC treatment.

FIG. 11 shows exemplary depletion of NK macrophages, NK or CD8+ cells has no effect on Ras/Myc-induced tumors, related to FIG. 2C. (A) The scheme of experimental procedure for cell depletion. Liver tumors were induced by Ras/Myc via HTVi. Mice were i.p. injected of NK1.1 Ab (600 μg) at day −11, clondronate liposome (200 μl) at day −11, and CD8 Ab (200 μg) at day −11, −6, −1, 4, and 9. PolyIC (4 μg/g) (or PBS) was i.p. injected at day −10, −8, −6, −4, −2. All mice were sacrificed (SAC) at 6 weeks for analysis. (B) Tumor burdens in the PBS-treated groups were calculated by liver weight/body weight ratios, maximal diameters of nodules (mm) and nodule numbers, to evaluate the effects of depleting NK cells, macrophages or CD8 T cells, without polyIC treatment. Data are represented as means±SD (n=5-7).

FIG. 12 shows exemplary induction of PD-L1 expression by polyIC in other APCs. (A) Flow cytometry analysis was performed to assess PD-L1 expression levels in DCs, macrophages, and MDSCs, respectively, in livers of the four groups of mice, as shown in FIG. 2A. (B) Quantification of mean fluorescence intensity (MFI) of PD-L1 expression in DCs, macrophages, and MDSCs, respectively, in livers, related to panel A.

Data in (B) are represented as means±SD (n=6).

FIG. 13 shows exemplary polyIC sensitizes PD-L1 blockade on the treatment of c-Met/b-catenin-induced HCC in mouse. (A) The scheme of experimental procedure for polyIC and/or PD-L1 Ab treatment. Mice were transfected via HTVi with human c-Met (0.5 μg/g), β-catenin (0.5 μg/g) and Sleeping Beauty (0.04 μg/g) expression constructs were at day 0. PolyIC (4 μg/g) (or PBS) was i.p. injected at day 42, 44, 46, 48, and 50, and PD-L1 Ab (or isotype IgG) was i.p. injected at day 45, 47, and 49. All mice were sacrificed (SAC) 8 weeks after oncogene injection. (B) Representative macroscopic views and H&E staining of liver sections in mice of control, polyIC, anti-PD-L1 and combination treatment. Magnification: ×20; Scale bar: 50 μm. (C) Tumor burdens were calculated by liver weight/body weight ratios, maximal diameters of nodules (mm) and nodule numbers in the four groups. Data in (C) are represented as means±SD (n=5). *p<0.05, for any other groups versus control.

FIG. 14 shows exemplary flow cytometry analysis of the representative cell number ratio, related to FIGS. 5 and 6. (A) Gating strategies as in FIG. 5C, Macrophage: CD11b+F4/80+; NK: CD4−nk1.1+; DC: CD11c+MHC II+; MDSC: CD11b+Gr1+. (B) Gating strategies as in FIG. 5D, CD8+ T cell: CD4-CD8+; CD4+ T cell: CD8−CD4+; regulatory T cell: CD4+foxp3+; B cell: CD4−CD45R+. (C) CD8+ cells were gated and further gating strategies as in FIG. 6A: cell proliferation: Ki67+; cell activation: CD44+CD62L−; cell cytotoxic function: granzyme B+.

FIG. 15 shows exemplary RNA-sequencing data analysis. RNA-seq analysis was performed for CD45+ cells isolated from livers of the four groups of mice as shown in FIG. 5A. (A) Venn diagram shows the numbers of up- or down-regulated genes in different treatment groups, compared to the control. (B) The top canonical pathways identified by the 1654 altered genes in the combination group by GSEA analysis. (C) Heat map shows the relative expression of key genes involved in the IFNγ signaling pathway and immune cells' cytotoxic effects. The red color indicates increasing expression with the green for decreasing expression.

FIG. 16 shows exemplary effects of polyIC and/or PD-L1 treatment on CD4 T cell, B cell, macrophage and neutrophil in the liver. The experimental procedure was same as in FIG. 4A. (A) Top: representative immunostaining of CD4. Bottom: quantification of CD4 T cell numbers per field. (B) Top: representative immunostaining of B220. Bottom: quantification of B220+ B cell numbers per field. (C) Top: representative immunostaining of F4/80. Bottom: quantification of macrophage percentages per field. (D) Top: representative immunostaining of Ly6G. Bottom: quantification of neutrophil numbers per field. Data in (A-D) are represented as means±SD (n=4-5).

FIG. 17 shows an exemplary therapeutic effect of the combination of polyIC+PD-L1 Ab in metastasized liver tumor model. MC-38 colon cancer cells were injected into the spleen and metastasized into the liver to grow tumors. Mice were treated with polyIC, PD-L1 Ab or polyIC+PD-L1 Ab as shown. (A) Experimental design. (B) Liver morphology. (C) Liver versus weight ratios. (D) Survival curve of mice.

FIG. 18 shows exemplary combination of polyIC+PD1 Ab (pIC+PD1) or polyIC and anti-CTLA4 Ab (pIC+CTLA4). (A) Experimental design. (B) Survival curve of mice treated with different combinations as shown. (C) General liver tumor morphology and Haemotoxylin and Eosin (H&E) staining of liver sections.

FIG. 19 shows exemplary therapeutic effect of polyIC+PD-L1 Ab in a NAFLD-HCC (Non-alcoholic fatty liver disease-hepatocellular carcinoma) tumor model induced by c-MET and PIK3CA, two oncogenes that are highly implicated in liver tumorigenesis in humans. (A) Experimental design. (B) Liver tumor phenotypes of mice sacrificed at 12 weeks. (C) General liver tumor morphology and Haemotoxylin and Eosin (H&E) staining of liver sections.

### DETAILED DESCRIPTION

Immunotherapy with checkpoint inhibitors for liver cancer, while in many clinical trials worldwide, may have uncertain outcomes given the unique immunotolerant microenvironment in the liver. Unexpectedly, a synthetic double stranded RNA (dsRNA), polyinosinic-polycytidylic acid (polyIC), was previously identified to have a potent liver tumor-inhibitory role. Injection of polyIC at the pre-cancer stage effectively prevented liver tumor initiation in several mouse models. However, injection of polyIC after tumor initiation showed no inhibition on tumor progression.

Provided herein are combination immunotherapies and methods of use for cancer treatment, in particular for the treatment of early and late stage liver cancer. In some embodiments, polyIC given at the pre-cancer stage effectively prevented liver tumorigenesis by activation of Natural Killer (NK) cells and macrophages, with no inhibition on tumor progression if injected after tumor initiation.

In some embodiments, polyIC administration potently induces PD-L1 expression in liver sinusoid endothelial cells. In some embodiments, a combination immunotherapy of polyIC and anti-PD-L1 antibody (PD-L1 blockade) effectively suppressed HCC progression in animal models. In some embodiments, polyIC sensitized hepatic response to PD-L1 blockade, resulted in sustained accumulation of active CD8+ cytotoxic T cells, robust tumor suppression and survival advantage. In some embodiments, these preclinical data may be instrumental for design of combination therapy for HCC, using polyIC and PD-L1 Ab or other similar reagents that can boost both innate and adaptive immunity.

In some embodiments, injection of polyIC or similar reagents may prevent liver tumorigenesis in subjects with chronic liver diseases, combination of polyIC and PD-L1 blockade may be an efficient immunotherapy for liver cancer.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of an ingredient being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing”, as used herein, is the prevention of the onset, recurrence or spread, in whole or in part, of the disease or condition as described herein, or a symptom thereof.

The terms “subject”, “patient”, or “individual”, as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the patient is a human. In some embodiments, the subject has experienced and/or exhibited at least one symptom of the disease, disorder, or condition to be treated and/or prevented.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein. The term “fixed combination” means that the active ingredients, e.g. a dsRNA and an antibody, are both administered to a patient simultaneously in the form of a single entity or dosage. The term “non-fixed combination” means that the active ingredients, e.g. a dsRNA and an antibody, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Exemplary Delivery Vehicles

Although the double stranded nucleic acid molecules may be delivered without a delivery vehicle, in certain embodiments, a delivery vehicle may be employed. Biomaterials, e.g., liposomes, hydrogels, or other materials formed of synthetic polymers or natural polymers, some of which may form micro- or nanoparticles, may be used as delivery vehicles. Numerous synthetic polymers have been used including polystyrene, poly-1-lactic acid (PLLA), polyglycolic acid (PGA) and poly-dl-lactic-co-glycolic acid (PLGA).

Another approach is the use of biological materials. Biological materials such as collagen, various proteoglycans, alginate-based substrates and chitosan. Collagen and collagen-GAG (CG) may be altered through physical and chemical cross-linking. Collagen-hydroxyapatite (CHA), collagen-hydroxy apitite (CHA) may be useful. Suitable biocompatible materials for the polymers include but are not limited to polyacetic or polyglycolic acid and derivatives thereof, polyorthoesters, polyesters, polyurethanes, polyamino acids such as polylysine, lactic/glycolic acid copolymers, polyanhydrides and ion exchange resins such as sulfonated polytetrafluorethylene, polydimethyl siloxanes (silicone rubber) or combinations thereof.

In one embodiment, the polymer is formed from natural proteins or materials which may be crosslinked using a crosslinking agent such as 1-ethyl-3-(3-dimethylamino-propyl)carbodiimide hydrochloride. Such natural materials include albumin, collagen, fibrin, alginate, extracellular matrix (ECM), e.g., xenogeneic ECM, hyaluronan, chitosan, gelatin, keratin, potato starch hydrolyzed for use in electrophoresis, and agar-agar (agarose), or other “isolated materials”. An “isolated” material has been separated from at least one contaminant structure with which it is normally associated in its natural state such as in an organism or in an in vitro cultured cell population.

Other biocompatible materials include synthetic polymers in the form of hydrogels or other porous materials, e.g., permeable configurations or morphologies, such as polyvinyl alcohol, polyvinylpyrrolidone and polyacrylamide, polyethylene oxide, poly(2-hydroxyethyl methacrylate); natural polymers such as gums and starches; synthetic elastomers such as silicone rubber, polyurethane rubber; and natural rubbers, and include poly[α(4-aminobutyl)]-1-glycolic acid, polyethylene oxide, polyorthoesters, silk-elastin-like polymers, alginate, EV Ac (poly(ethylene-co-vinyl acetate), microspheres such as poly (D, L-lactide-coglycolide) copolymer and poly (L-lactide), poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysinepolyethylene glycol, an agarose hydrogel, or a lipid microtubule-hydrogel.

In one embodiment, the delivery vehicle material includes but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, Nisopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(Llactide-co-glycolide, polyglycolide and poly(D,L-lactide), pol(D,L-lactidecoglycolide), poly(lactic acid colysine) and polycaprolactone.

In one embodiment, the biocompatible material for the distinct polymer is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (pcarboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the delivery vehicle may be formed of any of a wide range materials including polymers, including naturally occurring polymers, synthetic polymers, or a combination thereof. In one embodiment, the delivery vehicle comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acidpara-dioxanone-polyethylene glycol block copolymer, polypropylene, poly(ethylene terephthalate), poly(tetrafluoroethylene), poly-epsilon-caprolactone, or crosslinked chitosan hydrogels.

### Pharmaceutical Compositions

Pharmaceutical compositions having one or more of compounds comprising double stranded nucleic acid, e.g., double stranded RNA, suitable for administration, e.g., nasal, parenteral or oral administration, such as by intravenous, intramuscular, topical or subcutaneous routes, or by any other route of administration that allows drug to be delivered to the body or specific organs and tissues of the body, such as delivery to the liver, optionally further comprising sterile aqueous or non-aqueous solutions, suspensions, and emulsions. The compositions can further comprise auxiliary agents or excipients, as known in the art. The composition having one or more of the compounds described herein is generally presented in the form of individual doses (unit doses).

Preparations for parenteral administration include sterile aqueous or nonaqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the 5 inert diluents, such compositions can also include adjuvants, wetting agents, emulsifying and suspending agents, or sweetening, flavoring, or perfuming agents.

When a composition having one or more of the compounds described herein is used for administration to an individual, it can further comprise salts, buffers, adjuvants, or other substances which are desirable for improving the efficacy of the composition.

In one embodiment, the pharmaceutical composition is part of a controlled release system, e.g., one having a pump, or formed of polymeric materials (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger & Peppas, J. Macromol. Sci. Rev. Macromol. Chem., 23:61 (1983); see also Levy et al., Science, 228:190 (1985); During et al., Ann. Neurol., 25:351 (1989); Howard et al., J. Neurosurg., 71:105 (1989)). Other controlled release systems are discussed in the review by Langer (Science, 249:1527 (1990)).

The pharmaceutical compositions having one or more of compounds comprising double stranded nucleic acid comprise a therapeutically effective amount of the compound(s), for instance, those identified by screening methods, and optionally a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeiaes for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. These compositions can take the form of solutions, suspensions, emulsion, tablets, pills, capsules, powders, sustained-release formulations and the like. These compositions can be formulated as a suppository. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective, together with a suitable amount of carrier so as to provide the form for proper administration to the patient. The formulation should suit the mode of administration.

The compositions may be systemically administered, e.g., orally, in combination with a pharmaceutically acceptable vehicle such as an inert diluent. For oral administration, the compound(s) may be combined with one or more excipients and used in the form of ingestible capsules, elixirs, suspensions, syrups, wafers, and the like. Such compositions should contain at least 0.1% of active compound. The percentage of the compositions and preparations may, of course, be varied and may conveniently be between about 2 to about 60% of the weight of a given unit dosage form. The amount of active compound in such useful compositions is such that an effective dosage level will be obtained.

The compositions may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. Various other materials may be present. For instance, a syrup or elixir may contain the compound, sucrose or fructose as a sweetening agent, methyl and propylparabens as preservatives, a dye and flavoring such as cherry or orange flavor. Of course, any material used in preparing any unit dosage form, including sustained-release preparations or devices, should be pharmaceutically acceptable and substantially non-toxic in the amounts employed.

The composition can also be delivered by intravenous, intraperitoneal, intraarterial infusion or injection, or any other route of administration where delivery of a liquid formulation is suitable or appropriate 5 for drug delivery. Solutions of the compound(s) can be prepared in water or a suitable buffer, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of undesirable microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of undesirable microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride.

Sterile injectable solutions are prepared by incorporating the compound(s) in the required amount in the appropriate solvent with various of the other ingredients enumerated above, as required, followed by irradiation, steam (heat) or filter sterilization or any other preparatory method that results in a formulation that is essentially free of bacterial and/or viral contamination.

Useful liquid carriers include water, alcohols or glycols or water-alcohol/glycol blends, in which the present compound(s) can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Useful dosages of the compositions can be determined by comparing their in vitro activity and in vivo activity in animal models.

### Nonalcoholic Fatty Liver Disease—Hepatoceullar Carcinoma (NALFD-HCC)

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and may ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl:S186-90). NAFLD plays a major role in the progression of cirrhosis and hepatocellular carcinoma (HCC), a cancer with one of the most frequent solid tumors and highest mortality rates worldwide.

A number of embodiments of the invention have been described. Nevertheless, it will be understood that various modifications may be made without departing from the spirit and scope of the invention.

### Examples

### Materials and Methods

Mice and tumor models. All animals in this study were wild-type C57BL/6J mice from Jackson Laboratory, and male mice at age of 7-9 weeks were used for the experiments. The animal protocols (S09108) were approved by the Institutional Animal Care and Use Committee (IACUC) of the University of California San Diego, following National Institutes of health guidelines. Mouse liver tumors were induced by hydrodynamic tail vein injection (HTVi). The plasmids (PT3-EF1a-C-Myc; PT/Caggs-NRas-V12; PT3-EF1a-c-Met; pT3-EF1a-N90-β-catenin; pCMV-SB11) were gifts from Dr. X Chen at UCSF. The plasmid of GFP vector control (PT3-EF1a-EGFP) was constructed by cloning. All plasmid DNAs were diluted in PBS and injected at 0.1 ml/g body weight through tail vein in 5-7 seconds.

Drug administration and cell depletion assays. PolyIC (GE healthcare) was injected intraperitoneally (i.p.) at 4 mg/kg every other day for five doses at the indicated dates. Anti-mouse PD-L1 Ab (BE0101, Bioxcell) was i.p. injected at 200 μg (or 200 μg of rat IgG2b, BE0090, Bioxcell, as isotype control) every other day for three doses at the indicated dates. For NK cell depletion, mice were i.p. injected with 600 μg of NK1.1 Ab (BE0036, Bioxcell) (or 600 μg of mouse IgG2a (BE0085, Bioxcell) as isotype control) one time at the indicated date. Macrophages were depleted by i.p. injection of 200 μL of clondronate liposome (C09T0317, www.liposome.com) (or 200 μL of PBS control liposome (P08T0317, www.liposome.com) as isotype control) one time at the indicated date. CD8 T cells were depleted by i.p. injection of 200 μg of anti-mouse CD8α (BE0061, Bioxcell) (or 200 μg of rat IgG2b (BE0090, Bioxcell) as isotype control) three or five times at the indicated dates.

Flow cytometric analysis. After liver perfusion, mouse NPCs were isolated and subjected to FACS analysis. The following mAbs were used: CD62L (11-0621-82), CD4 (45-0042-82), CD44 (25-0441-82), Ki67 (51-5698-82), foxp3 (48-5773-82), CD8a (12-0081-82), CD4 (11-0041-82), CD45R (45-0452-82), PD-1 (25-9985-82), Gr1 (11-5931-82), CD11b (45-0112-82), MHC II (25-5321-82), CD11c (48-0114-82), granzyme B (50-8898-82) were all from eBioscience (San Diego, USA). CD8a (100759), PD-L1 (124308), F4/80 (123115), CD45 (103107), CD146 (134713), NK1.1 (108717) were from Biolegend (San Diego, USA). To exclude dead cells, cells were firstly incubated by Ghost Dye™ Red 780 Ab (13-0865-T100, Tonbo). Flow cytometric analysis was conducted on a LSRFortessa™ X-20 (BD Bioscience) and FlowJo software (Tree Star, Ashland, Oreg., USA).

RNA sequencing and bioinformatic data analysis. Immune cells were isolated from mouse liver and total RNAs were extracted using RNeasy Microarray Tissue Mini Kit (QIAGEN #73304). cDNA libraries were prepared using Illumina TruSeq Stranded mRNA Library Prep Kit (RS-122-2101, Illumina). RNA-sequencing (RNA-seq) was performed using Illumina HiSeq 4000 at the IGM Genomics Center, UCSD. RNA-seq generated raw data were aligned to the GRCm38 mouse reference genome using Star program (2.3.0). Gene differential expression analysis was performed using Cuffdiff to obtain the expression levels of genes in each sample. The significant differences in gene expression were based on q values (<0.05) and fold change (>2). Gene Set Enrichment Analysis (GSEA) was performed for pathway analysis online (software.broadinstitute.org/gsea). Heatmaps were generated using the heatmap package using R program.

Statistical analysis. Statistical analysis was done using GraphPad Prism 9.9.6. Values were presented as means±SD. Statistical significance between means was performed using Student's T-Test. P value<0.05 was considered significant (*p<0.05, **p<0.01).

Histopathology and immunostaining. Liver tissues were obtained and fixed in Z-fix solution (Anatech) for later paraffin embedding or directly embedded in Tissue-Tek

OCT compound (Sakura Finetek) for frozen sectioning. Hematoxylin and eosin (H&E) stained paraffin sections were processed for histopathological evaluation of hepatocellular carcinoma (HCC). Paraffin sections were also stained for Ki67 (14-5698-80, eBioscience), CD45 (103106, Biolegend), CD8 (14-0808-80, eBioscience), CD4 (41-9766-80, eBioscience), B220 (14-0452-81, eBioscience), F4/80 (14-4801-81, eBioscience), Ly6G (14-5931-81, eBioscience) and TUNEL assay, according to the manufacturers' procedures. Frozen sections were fixed with acetone overnight, stained for PD-L1 (BE0101, Bioxcell) and VE-Cadherin (AF1002, R&D) and secondary antibodies, and finally counterstained with Vectshield mounting medium with DAPI (H-1200, VWR), according to the standard protocols. The immunostaining images were acquired using the microscope (Olympus IX71) and the matched CellSense software.

Immunoblotting and quantitative real-time PCR. Immunoblotting and real-time quantitative PCR (qRT-qPCR) were performed according to the standard protocols. Antibody against PD-L1 was from Bioxcell and GAPDH were from Cell signaling.

Total RNAs were extracted from fresh liver tissues and purified using the TRIzol reagent (Ser. No. 15/596,018, Thermo Fisher Scientific). RNAs were reverse transcribed into cDNA using High-Capacity cDNA Reverse Transcription Kit (4368814, Thermo). Liver genomic DNAs were extracted using Genomic DNA Buffer Set (19060, QIAGEN). RT-PCR was performed with DyNAmo Flash SYBR Green qPCR Kit (F415, Thermo) using Mx3000P qPCR system (Agilent Technologies). Relative quantitation analysis was performed with reference to gapdh RNA using the comparative cycle threshold (CT) method. Each sample was detected for at least 3 duplicates. The primer sequences were listed as followed: gfp, Forward (5′-3′):

### CCGACCACATGAAGCAGCAC, Reverse (5′-3′):

TCGCCCTCGAACTTCACCTC; cd274, Forward (5′-3′):

### ATTGCTCCTTGACTGCTGGCTG, Reverse (5′-3′):

ATTGCTCCTTGACTGCTGGCTG; gapdh, Forward (5′-3′):

### CGACTTCAACAGCAACTCCCACTCTTCC, Reverse (5′-3′):

### TGGGTGGTCCAGGGTTTCTTACTCCTT.

Cell isolation and culture. Liver in-situ perfusion was performed with solution containing 0.08% collagenase I (17018029, Gibco). Isolated cells were centrifuged at

70 g for 5 min to collect the supernatant. Then non-parenchymal cells (NPCs) were centrifuged at 300 g for 30 min and red blood cell lysis was performed using ACK lysis buffer (1954589, Gibco). For isolation of immune cells, after incubation with CD16/32 Ab (BD) for Fc blockade, NPCs were isolated by magnetic beads separation (MACS) using PE-conjugated CD45 mAb (103106, Biolegend) and PE microbeads (130-048-801, Miltenyi Biotec). For LSEC isolation, NPCs were isolated by MACS using CD146 microbeads (130-092-007, Miltenyi Biotec). Then LSECs were seeded into collagen-coated 24-well plates at a density of 2×105/well, in DMEM medium (4500 mg/mL glucose) with 8% fetal bovine serum. After culturing for 72 hours, cells were treated by polyIC (80 μg/ml) for 24 hrs and harvested for analysis.

### Results

### PolyIC Inhibits Tumor Initiation but not Progression in the Liver

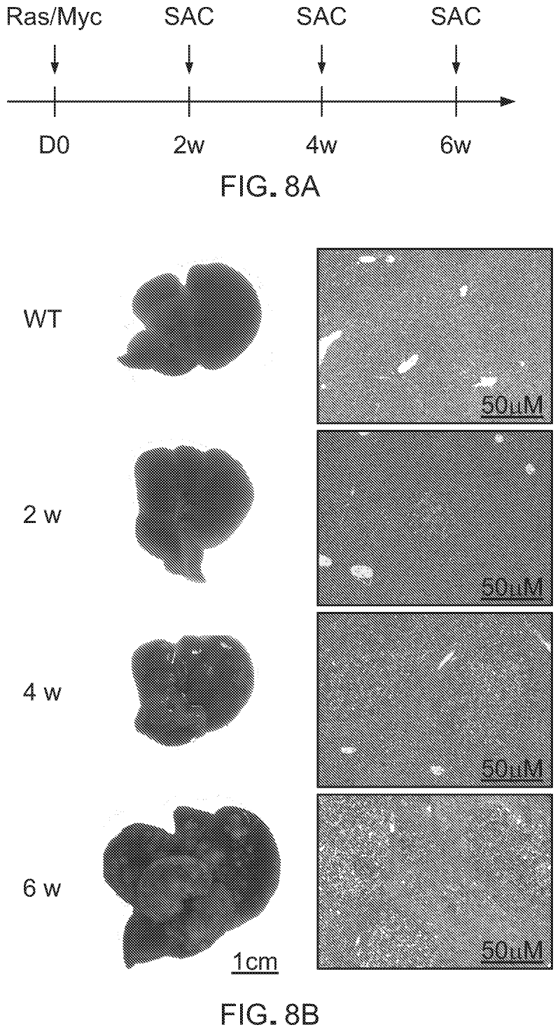

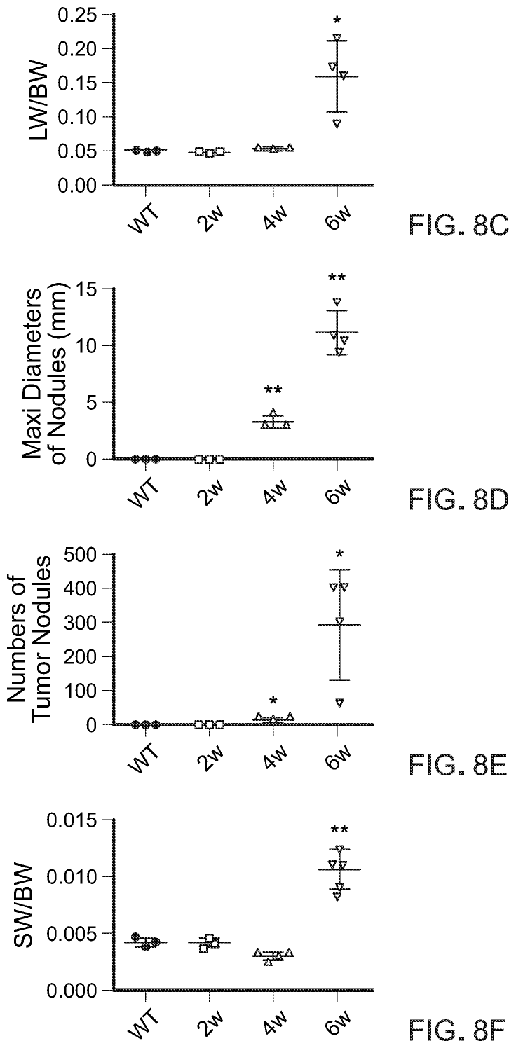

In previous experiments, it was found that polyIC, an inducer of the Mx1-Cre gene deletion system, had a potent tumor-inhibitory effect, irrespective of gene ablation by Mx1-Cre induction. Other mouse HCC models that are driven by hydrodynamic tail vein injection (HTVi) of oncogenes were examined. Two plasmids (Ras/Myc) were injected that express human N-Ras and human c-My c, together with a sleeping beauty transposase (FIG. 8A). Mice were sacrificed for phenotypic analysis at 2, 4, or 6 weeks, and liver tumor nodules were visible as early as 4 weeks and progressed rapidly, as examined macroscopically, H&E staining of liver sections (FIG. 8B), and statistical analysis of liver weight/body weight (LW/BW) ratios, maximal diameters and numbers of tumor nodules, as well as increased spleen/body weight ratios (FIG. 8C-F).

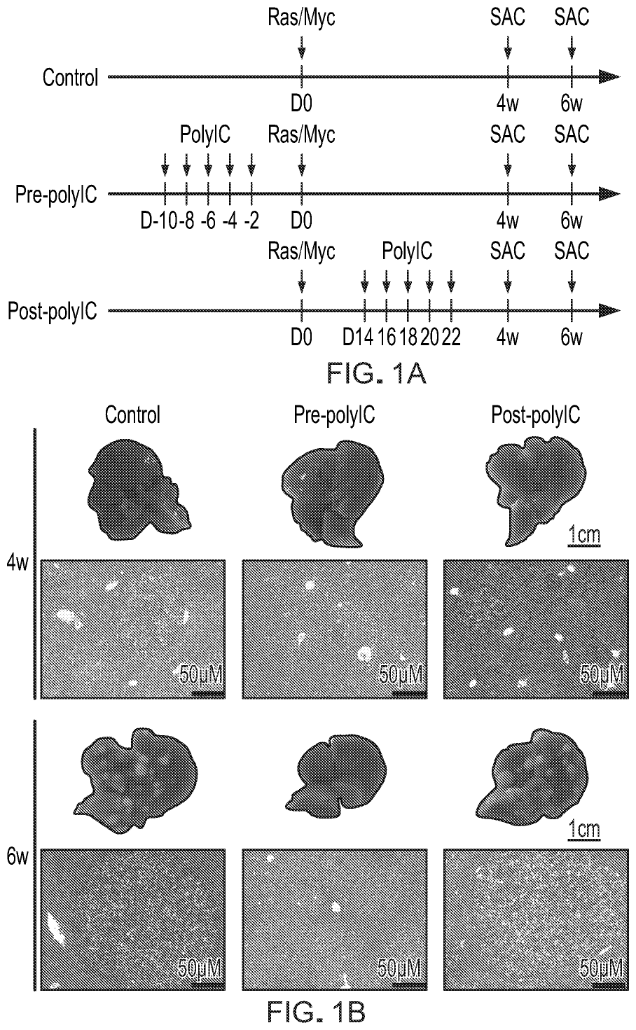

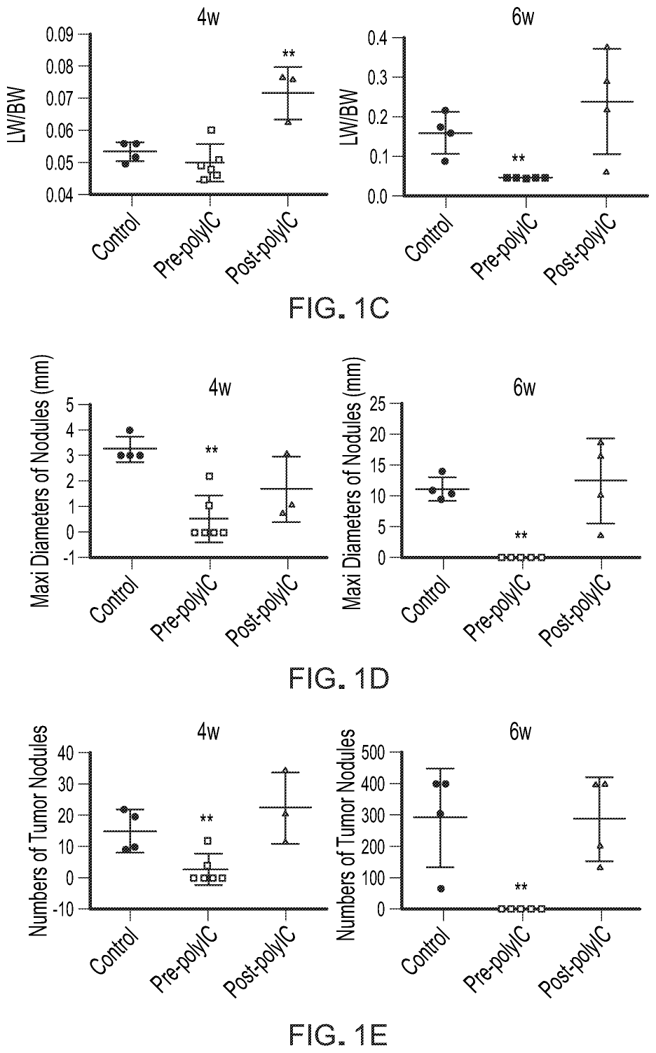

The tumor-suppressing activity was evaluated of polyIC injected before or after tumor induction by Ras/Myc. The synthetic dsRNA was injected i.p. every other day for a total of 5 doses and the tumor burdens were examined 4 and 6 weeks after oncogene transfection (FIG. 1A). PolyIC given before tumor initiation by oncogenes (pre-polyIC) significantly suppressed tumor formation, as determined by macroscopic and histological examination, LW/BW ratios, maximal diameters and numbers of tumor nodules (FIG. 1B-E). However, polyIC administration starting 2 weeks after oncogene transfection (post-polyIC) did not have significant inhibition on tumor burdens examined by these criteria (FIG. 1B-E). Further, the LW/BW ratios even increased significantly in the post-polyIC group, compared to the control. These results suggest that polyIC given at the pre-cancer stage can efficiently prevent initiation of liver tumors driven by the oncogenes, consistent to previous data showing a preventive role of the dsRNA in HCC induced by diethylnitrosamine (DEN), or HCC and ICC (intrahepatic cholangiocarcinoma) driven by Pten deletion and associated fatty liver disease. Notably, polyIC alone does not have therapeutic effect if given after tumor initiation as revealed in this and previous studies.

Activation of Innate Immunity is Required for the Tumor-Preventive Effect of polyIC

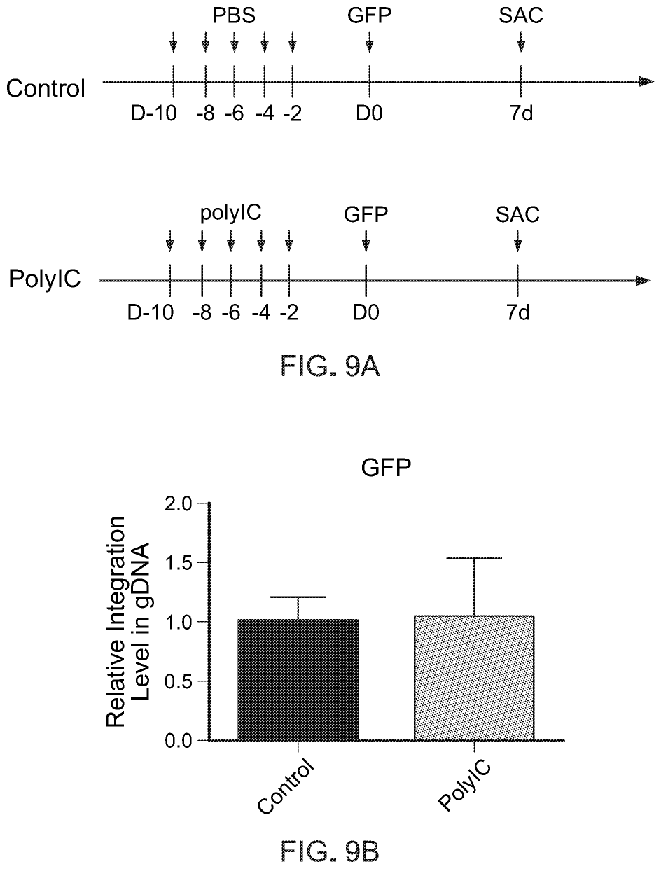

The mechanisms underlying polyIC's prevention of liver tumorigenesis induced by the oncogenes Ras/Myc was explored. First, it was investigated if pre-polyIC treatment had influenced genomic integration of the exogenous oncogenic cDNAs in hepatocytes, which is required for tumor induction using the HTVi approach. A plasmid expressing GFP was injected into mice via HTVi with or without pre-treatment of polyIC (FIG. 9A). Genomic DNAs were extracted from liver lysates 7 days later for quantitative PCR analysis, and similar levels of the GFP cDNA were detected between the two groups (FIG. 9B), suggesting no effect of polyIC pre-treatment on plasmid DNA transfection and integration into the hepatocyte genome.

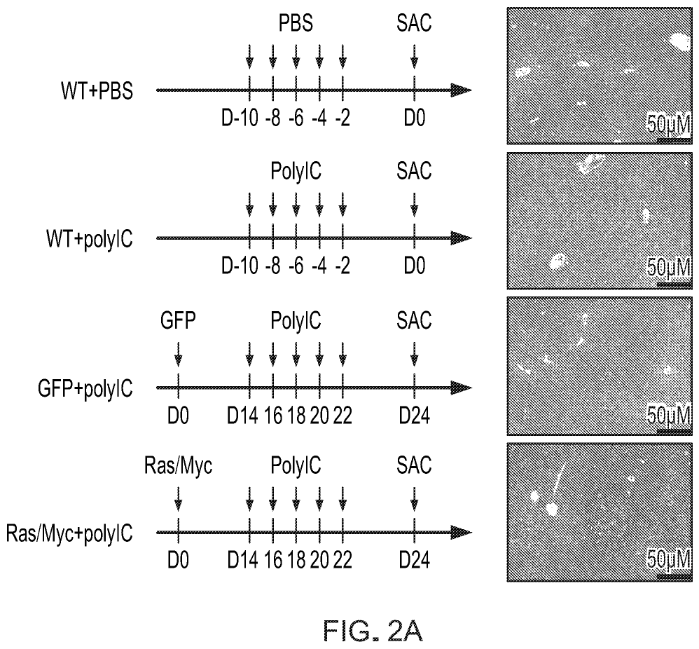

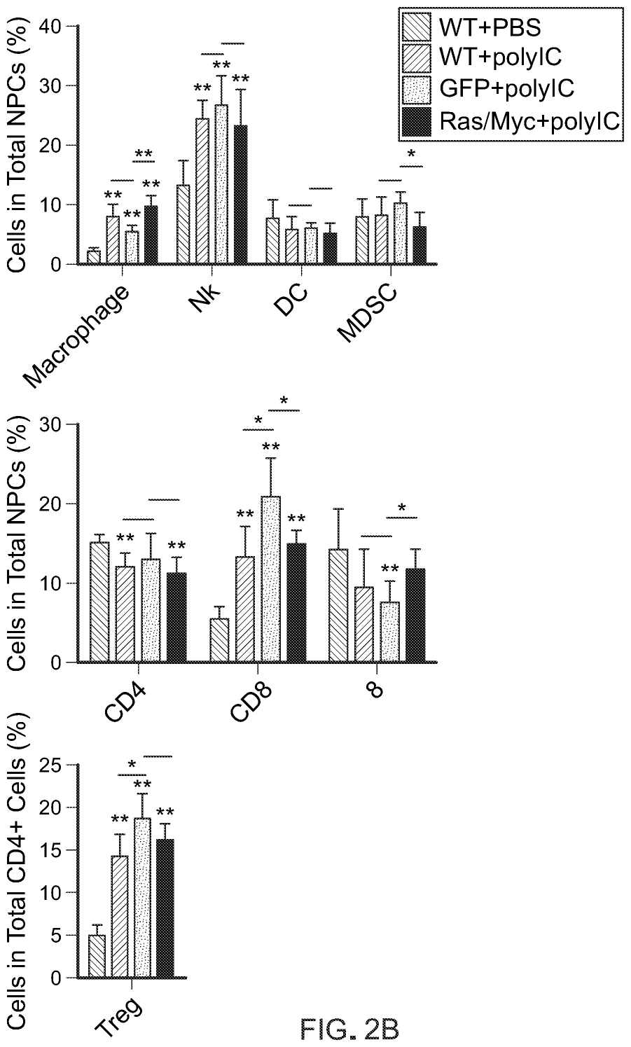

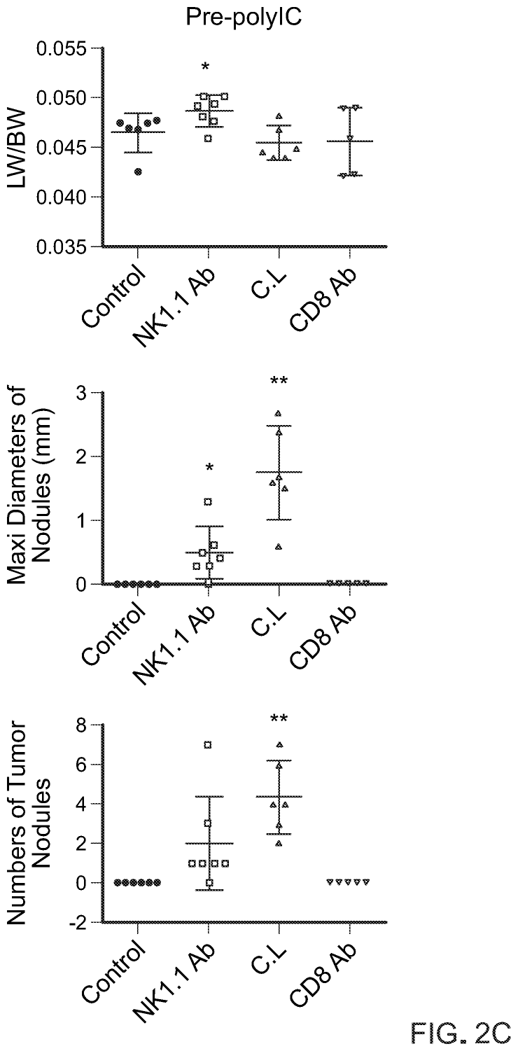

The effects of polyIC on various immune cell subsets under different conditions were interrogated (FIG. 2A). By comparing the WT livers with or without polyIC treatment, an impact of polyIC itself was determined, without liver damage caused by the HTVi procedure. Comparing the polyIC effects in livers that received GFP or Ras/Myc oncogenes, the influence of tumor development on the polyIC effect was evaluated. As shown in FIG. 2B, polyIC treatment induced significant increase of macrophages and NK cells, with a modest effect on myeloid-derived suppressor cells (MDSC) and no significant impact on dendritic cells (DC) in all three polyIC-treated groups, relative to the WT control. Further, polyIC injection boosted the numbers of CD8 T cells and regulatory T cell (Treg), with the CD4 T cells and B cells unchanged or modestly decreased (FIG. 2B). The changes in various cell subsets were quite similar among the three polyIC-treated groups, relative to the WT control (FIG. 2B), suggesting that polyIC modulation of immunity is independent of the hydrodynamic injection.

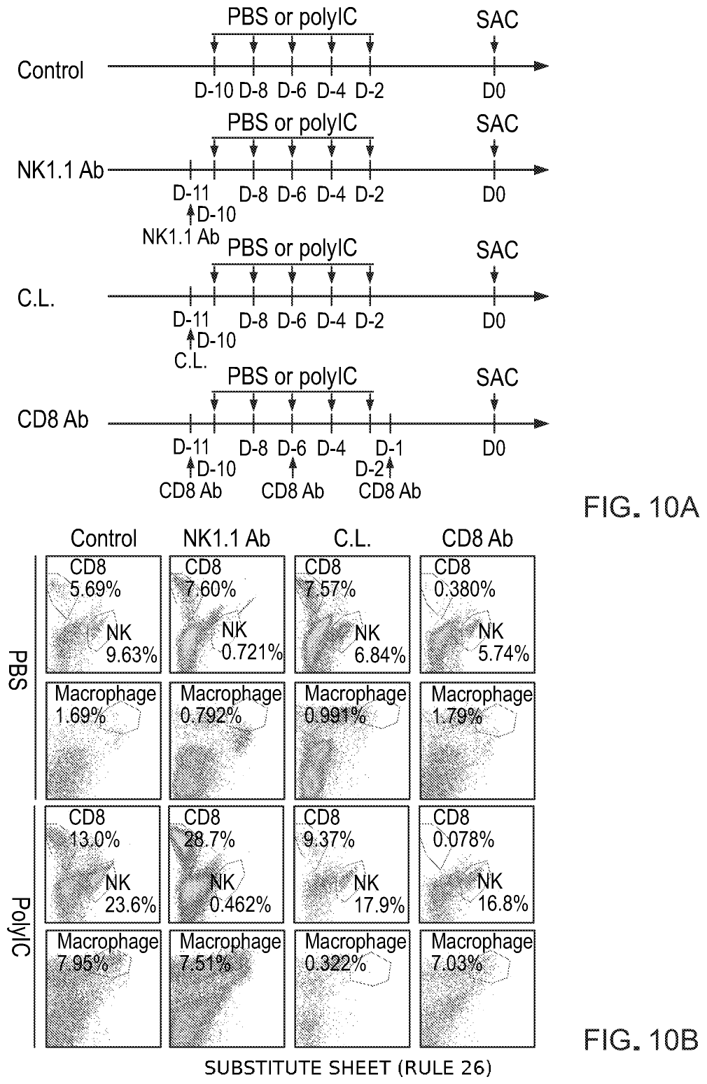

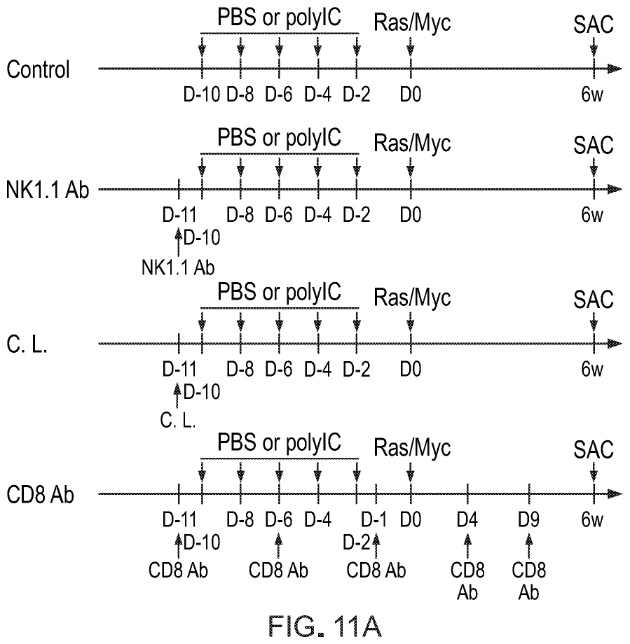

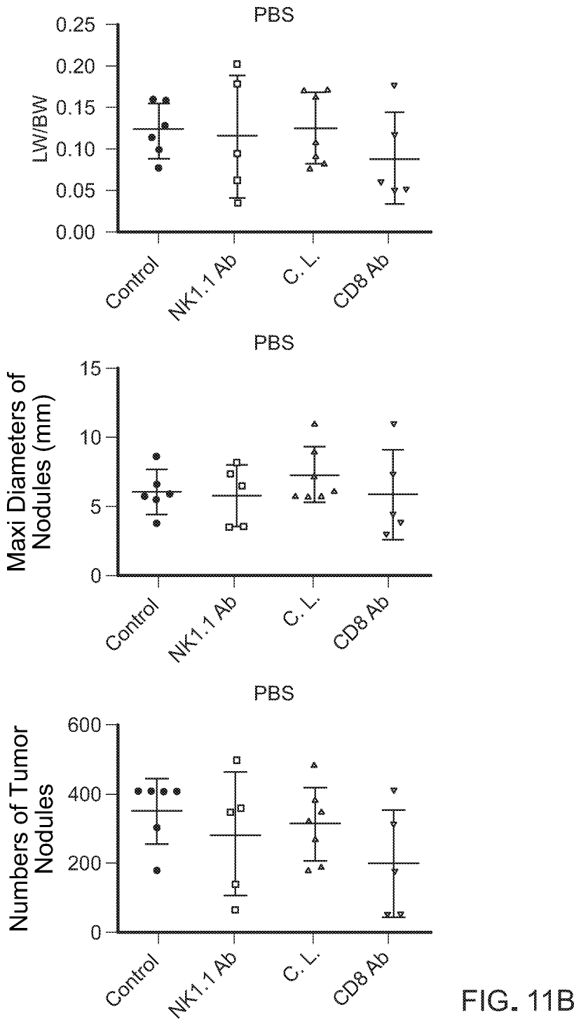

As the previous data suggested possible involvement of NK cells and macrophages in polyIC-mediated clearance of tumor-initiating cells (TICs), it was determined if these cell subsets were indeed responsible for polyIC's inhibition of HCC. An anti-NK1.1 antibody, clondronate liposome or anti-CD8 antibody was injected to deplete or block NK cells, macrophages or CD8+ T cells (FIG. 10A). At 2 days after last polyIC injection, liver NPC cells were isolated for FACS analysis to evaluate the depletion efficiency (FIG. 10A). The numbers of macrophages, NK and CD8 T cells decreased markedly after injection of these reagents, without or with polyIC injection (FIG. 10). It was then asked if depletion of these cell subsets had any impact on tumorigenesis, by examining the tumor loads 6 weeks after Ras/Myc transfection (FIG. 11A). In mice without pre-polyIC treatment, depletion of these cell subsets did not reduce significantly the tumor burdens (FIG. 11B). Therefore, the basal activities of NK cells, macrophages or CD8 T cells in the liver had little effect on liver tumorigenesis. However, depletion of NK cells or macrophages abrogated the tumor-inhibitory effect of pre-polyIC treatment, with no effect of CD8 T cell depletion observed (FIG. 2C). These data indicate that polyIC inhibition of HCC initiation is dependent on activation of NK cells and macrophages, but not CD8 T lymphocytes.

### PolyIC Upregulates PD-L1 Expression in Liver Sinusoid Endothelial Cells (LSEC)

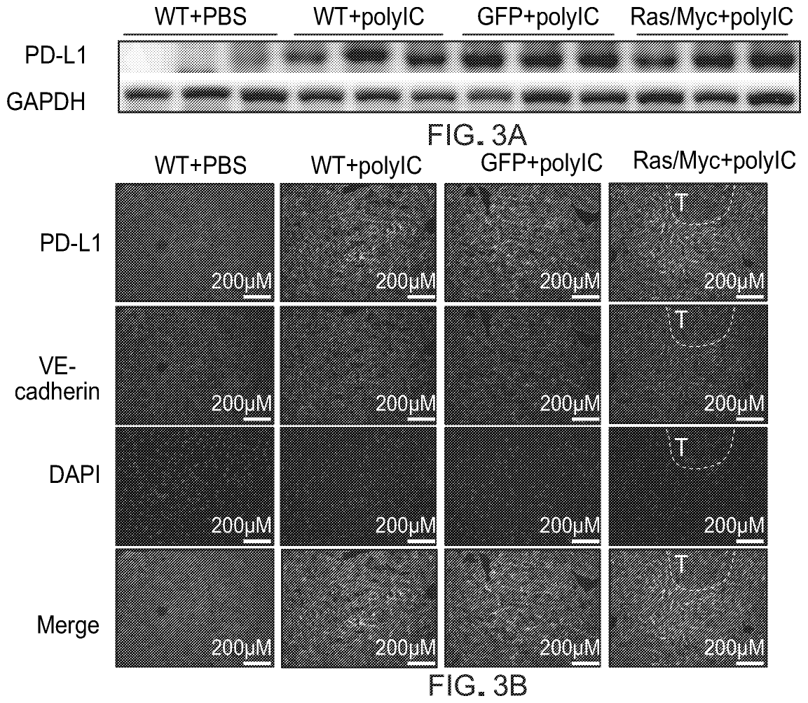

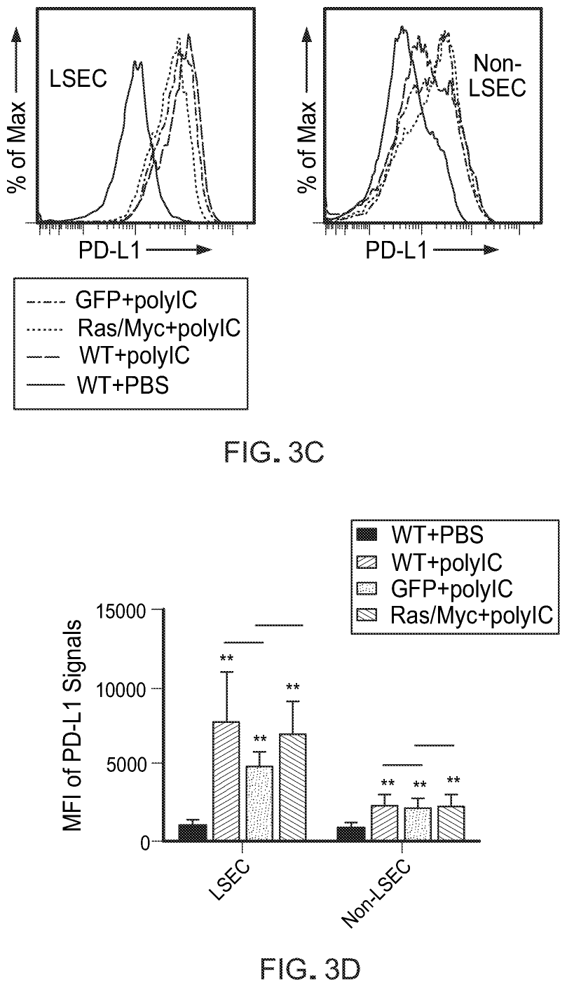

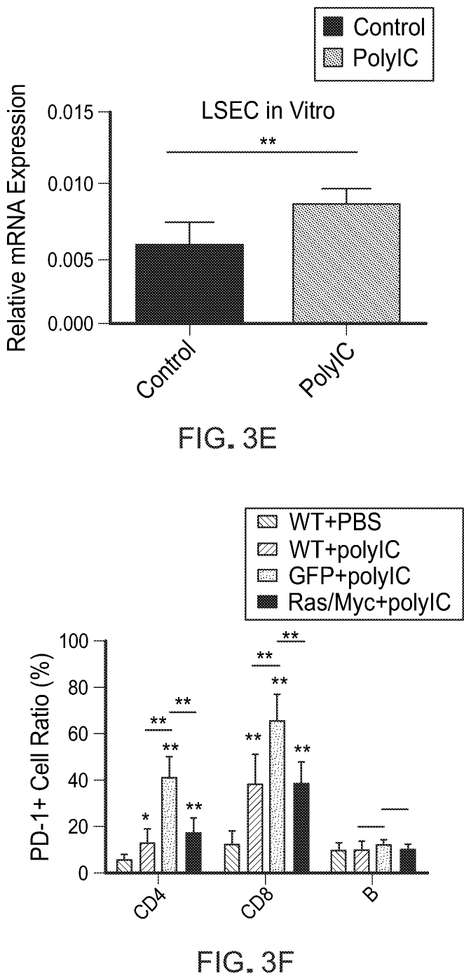

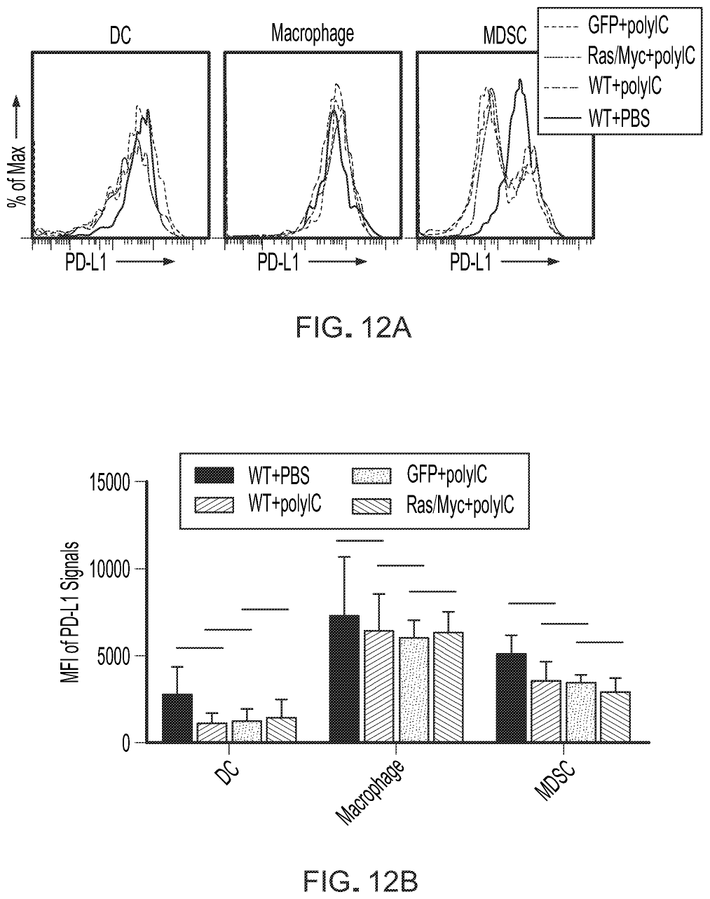

Previous RNA-seq analysis detected significant increase of CD274 (PD-L1) expression in poly-IC-treated livers. Consistently, immunoblotting detected high levels of PD-L1 protein contents in livers treated with polyIC, regardless of oncogene transfection (FIG. 3A). PD-L1 expression patterns were examined in the liver, and found that the PD-L1 signal was markedly elevated in liver sinusoid endothelial cells (LSEC), overlapping with the endothelial marker VE-cadherin (FIG. 3B), with no increase of PD-L1 expression in tumor areas in Ras/Myc-transfected livers. Flow cytometry showed dramatically increased PD-L1 expression in LSECs in all polyIC-treated groups, but not in other NPCs (non-LSEC), relative to the control (FIG. 3C). The mean fluorescence intensity (MFI) of PD-L1 expression was significantly increased in LSECs of all polyIC-treated groups, relative to the control, with only modest increase in non-LSECs (FIG. 3D). FACS analysis also showed similar low levels of PD-L1 expression in DC, macrophages and MDSC, which were not influenced dramatically by polyIC treatment (FIG. 12A-B), different from other reports showing polyIC upregulation of PD-L1 expression in antigen-presenting dendritic cells or epithelial cells.

To define a direct role of polyIC in PD-L1 induction, LSECs isolated from WT mouse livers were cultured. The PD-L1 mRNA level was significantly elevated in LSECs after polyIC treatment for 2 days (FIG. 3E), confirming upregulation of PD-L1 expression in LSECs by polyIC in the liver, independent of oncogene transfection or tumor development. PolyIC impact on PD-1 expression in infiltrated lymphocytes including CD4, CD8 T cells and B cells was also assessed. Following polyIC treatment, PD-1-positive cell ratios were higher in CD4 and CD8 T cells, but not in B cells, than the control (FIG. 3F). Thus, polyIC treatment likely induced T cell dysfunction at least in part by promoting PD-L1/PD-1 signaling in the liver microenvironment.

Combination of polyIC and PD-L1 Blockade Suppresses HCC Progression

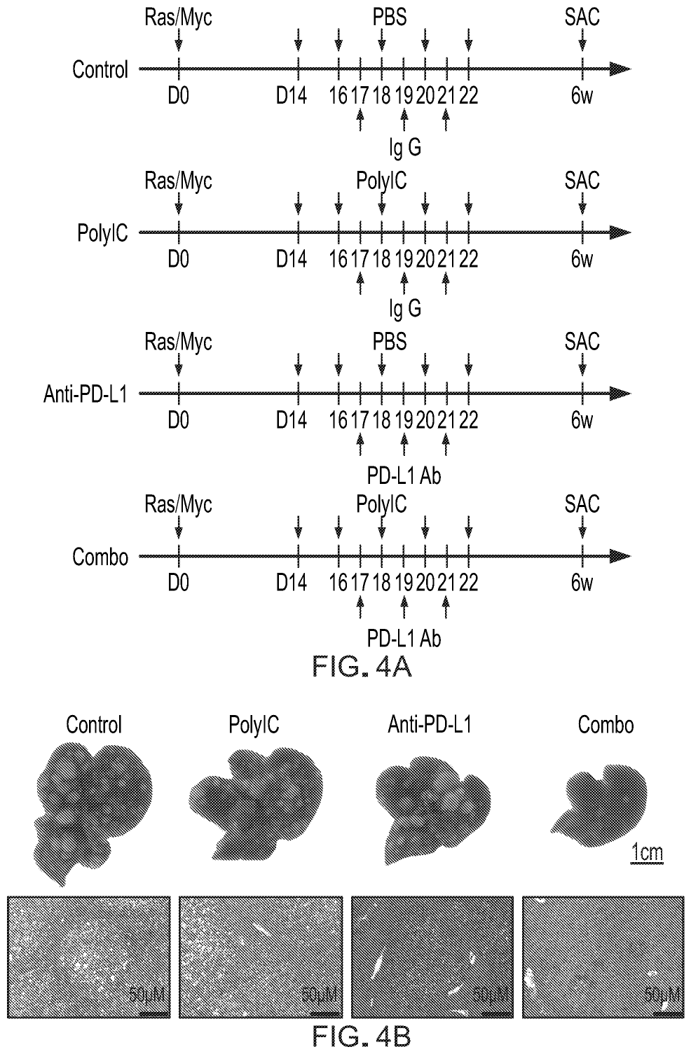

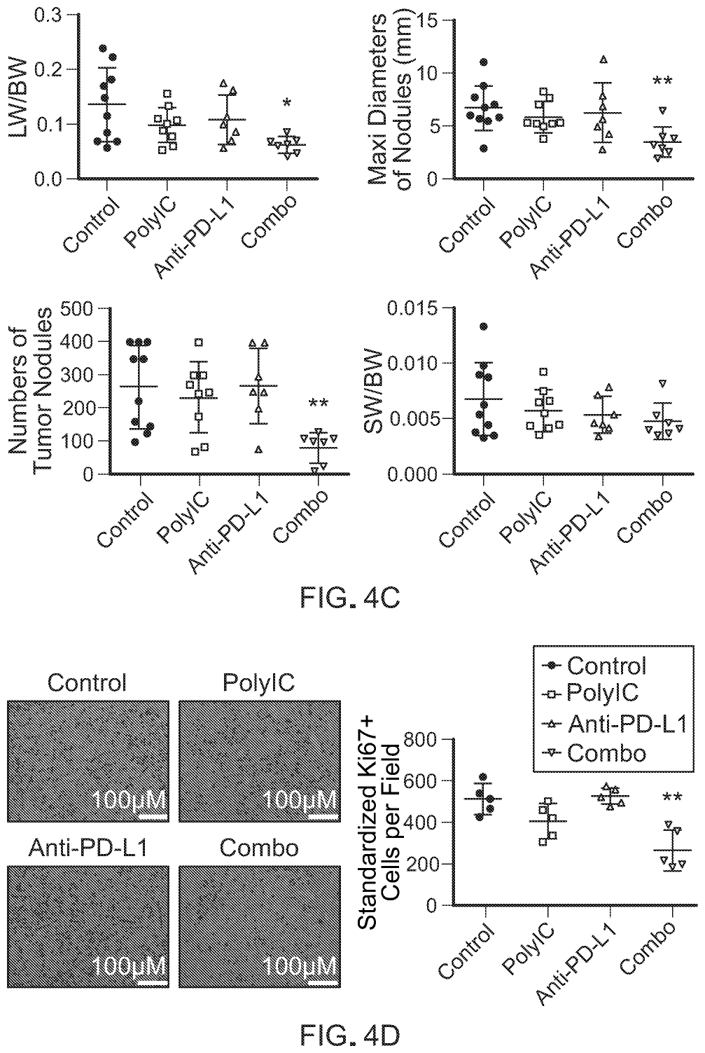

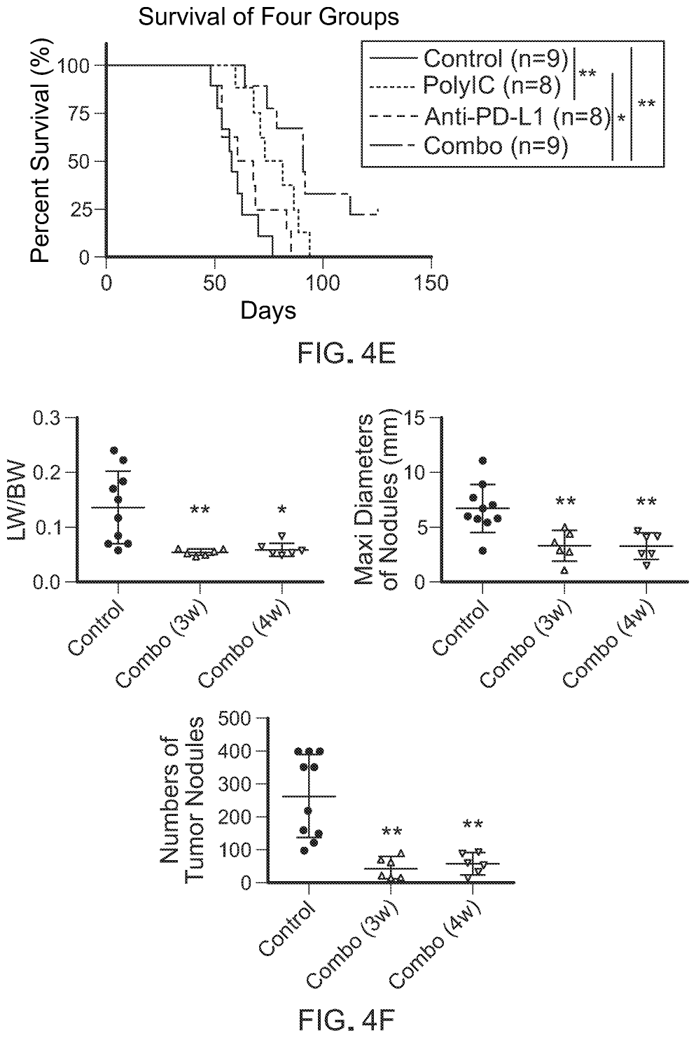

Given that PD-L1 expression is required for tumor response to anti-PD-1/PD-L1 treatment, the polyIC upregulation of PD-L1 expression in LSEC prompted an exploration of an immunotherapy for HCC by combined treatment of polyIC and PD-L1 Ab. The Ras/Myc-transfected mice were divided into four groups, and treated with polyIC, PD-L1 Ab or combination of polyIC and PD-L1 Ab (Combo). Based on the kinetics of tumor development in this model (FIG. 8), administration of the reagents was started 2 weeks after oncogene transfection, and examined the tumor burdens at 6 weeks (FIG. 4A). Injection of either polyIC or PD-L1 Ab failed to reduce the tumor loads, showing similar liver sizes and numbers of tumor nodule, relative to the control (FIG. 4B). However, combination of polyIC and PD-L1 Ab significantly suppressed tumor progression, by macroscopic view and H&E staining or evaluated by the liver/body weight ratios, sizes and numbers of tumor nodules (FIG. 4B-C).

Tumor cell proliferation was assessed, and found no significant changes in Ki67-positive cell ratios in tumor areas in the polyIC or anti-PD-L1 group, compared to the control. However, the combined treatment significantly decreased the Ki67-positive ratio in the tumor areas (FIG. 4D). Mouse survival analysis showed the median survival time 58.0±9.3, 77.5 11.8, 64.5±14.2 and 9127.0 days for the control, polyIC, anti-PD-L1 and the combination groups (FIG. 4E). Overall, the median survival time was similar between the anti-PD-L1 and control groups, with prolonged survival in polyIC-treated group. The combination treatment exhibited most significant extension of mouse survival, as compared to the other groups, with 2 mice still alive at the end time point of observation (18 weeks after oncogene injection). A therapeutic effect of the combination at later stages of tumor progression was also explored, with the treatment starting at 3 or 4 weeks after Ras/Myc transfection. Again, co-injection of polyIC and PD-L1 Ab significantly decreased tumor burdens, compared to the control (FIG. 4F).

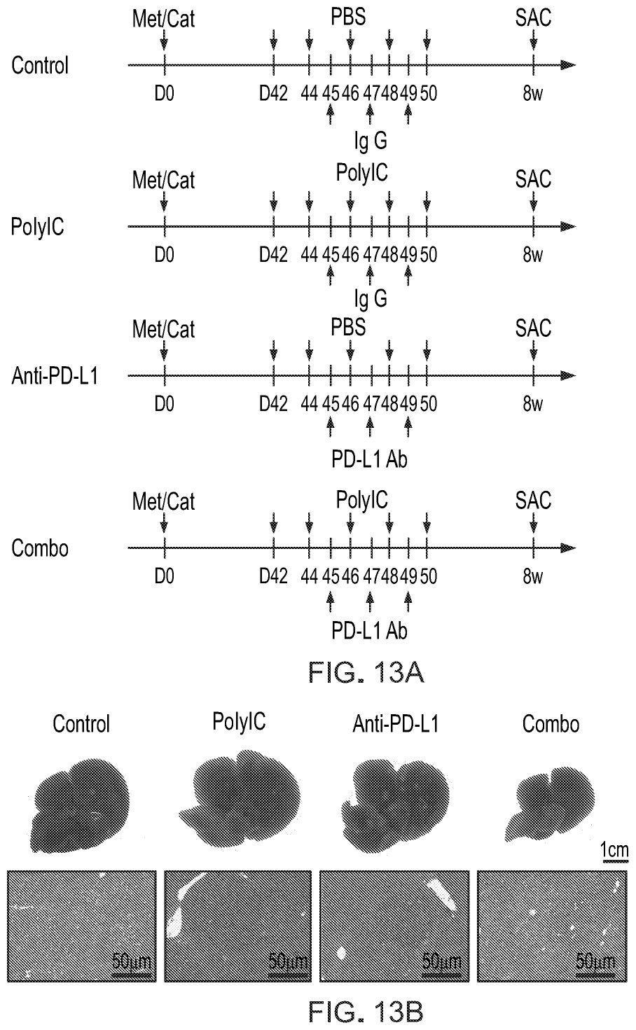

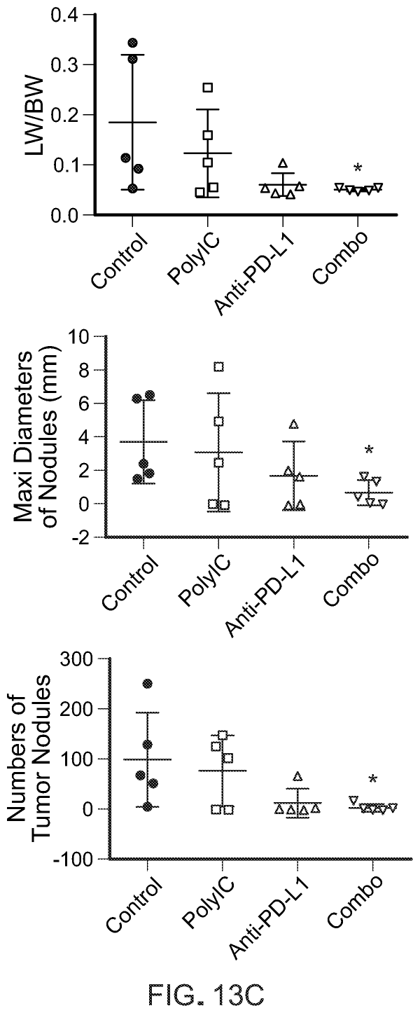

The combination therapy was tested in another HCC model by transfection of constructs (Met/Cat) expressing human c-MET and a truncated β-catenin mutant, two oncogenes that are frequently detected in human HCC patients. Based on the pathogenic process in this model, the treatment was started 6 weeks after c-MET/0-catenin transfection, and examined the tumor loads at 8 weeks (FIG. 13A). Macroscopic view showed that neither polyIC nor PD-L1 Ab inhibited HCC development in this model, while no tumor nodule was even visible in the combination group. H&E staining also showed similar results and more infiltrated mesenchymal cells in the liver treated with the combination (FIG. 13B). Statistical analysis showed a modest but insignificant decrease of tumor burdens following treatment of polyIC or anti-PD-L1 alone, compared to the control. However, all of the parameters to evaluate tumor burdens were significantly decreased in the combination group (FIG. 13C). Therefore, the combination of polyIC and PD-L1 blockade has a synergistic inhibitory effect on HCC progression in different animal models.

Sensitization of PD-L1 Blockade by polyIC Boosts Anti-Tumor Immunity in the Liver

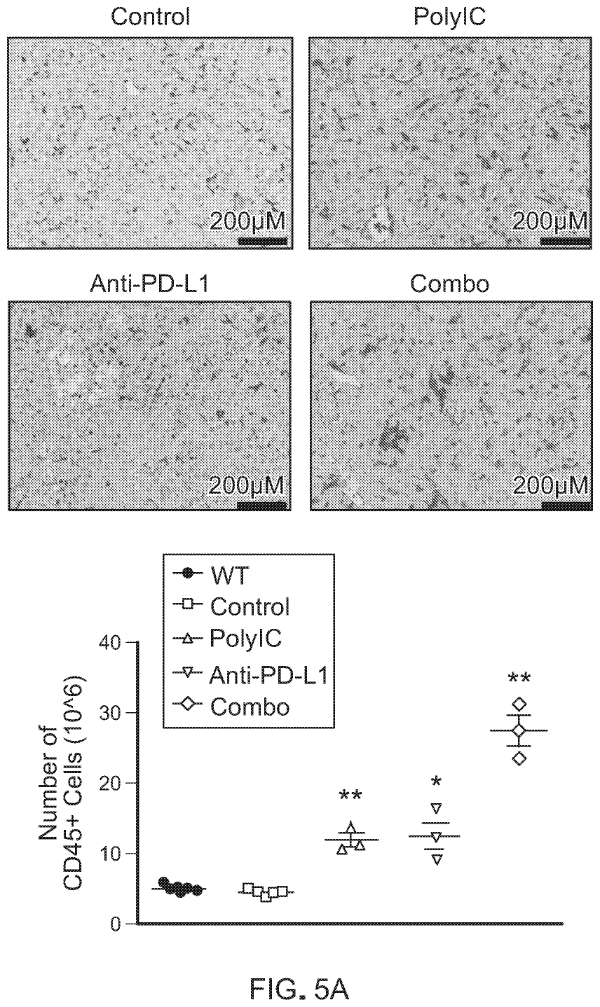

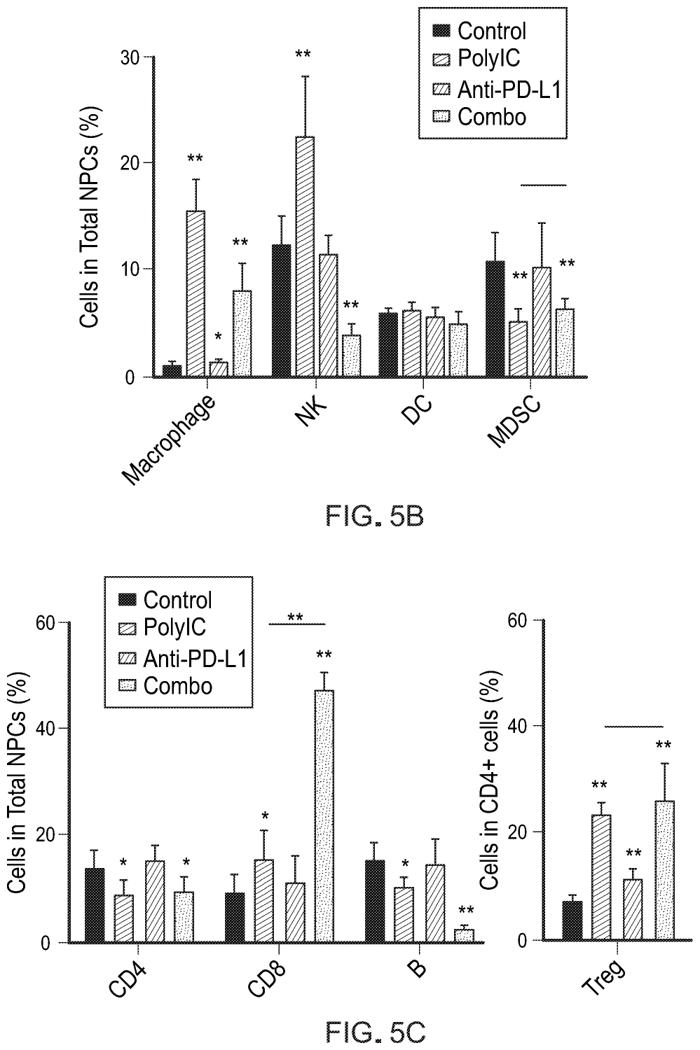

The underlying mechanisms for the potent tumor-inhibitory effect of the combination treatment was dissected. Ras/Myc-transfected mice treated with polyIC, PD-L1 Ab or the combo as in FIG. 4A were sacrificed 2 days after the last polyIC injection. Immunostaining demonstrated significantly increased infiltration of CD45+ cells into livers treated with polyIC or anti-PD-L1, but the combo caused even more infiltration of CD45+ cells into the liver (FIG. 5A, top). CD45+ cells were isolated and counted by also including WT livers without oncogene transfection. The numbers of total immune cells (4.510.51×10⁶) in untreated Ras/Myc transfected liver were similar to the WT liver (5.110.66×10⁶), polyIC or PD-L1 Ab treatment significantly increased the number of immune cells in livers to 11.92±1.52×10⁶ and 12.52±2.80×10⁶, respectively. However, the immune cell number was markedly increased to 27.27±3.05×10⁶ in livers treated with the combination (FIG. 5A, down).

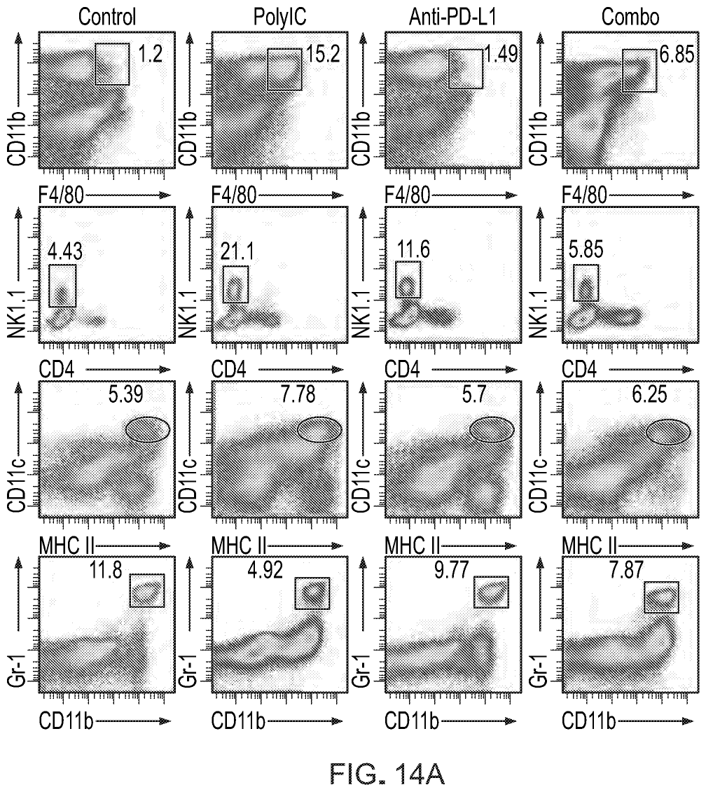

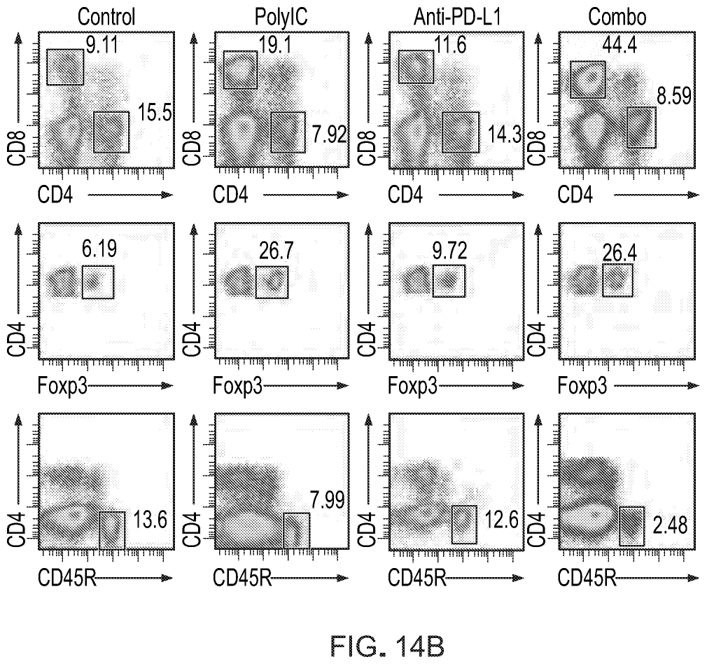

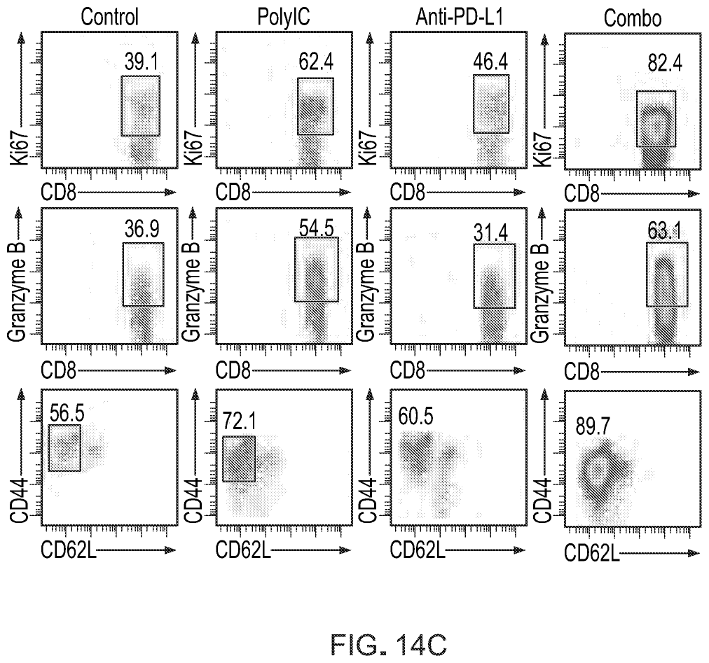

A more detailed FACS analysis of innate and adaptive immune cell subsets in the liver was performed. The relative cell numbers were calculated as ratios in the total numbers of NPCs, except the Treg cells that were calculated as a ratio to the total CD4+ T cells. Flow cytometry was performed on innate immune cells, including macrophages (CD11b+F4/80+), NK cells (CD4-NK1.1+), DC (MHC II+CD11c+) and MDSC (CD11b+Gr1+) (FIG. 14A). Compared to the control, polyIC injection significantly increased the numbers of macrophages and NK cells, which were not influenced by PD-L1 Ab treatment. The combination also increased the numbers of macrophages and NK cells significantly, but the stimulating effect was weaker than polyIC alone. There was no significant difference of the DC cell numbers between these groups. Treatment of polyIC alone or the combination had similar effects in decreasing the numbers of MDSC (FIG. 5B). Therefore, polyIC treatment had a significant impact on innate immunity in the liver, with no impact by concurrent injection of PD-L1 Ab.

Adaptive immune cell subsets were then analyzed, including conventional CD4 T cell (CD4+foxp3−), CD8 cytotoxic T cell (CD4−CD8+), Treg (CD4+foxp3+) and B cell (CD4-CD45R+), by flow cytometry (FIG. 14B). Treatment of polyIC alone or in combination with PD-L1 Ab caused similar decrease of CD4 lymphocytes, while PD-L1 Ab did not have impact on the CD4 cell ratio. Interestingly, the number of CD8 T cells was modestly increased in polyIC-treated liver, but was dramatically elevated to 46.85±2.84% of all NPCs in livers treated with the combination. The ratio of Treg cells in CD4 T cell pool was significantly increased in PD-L1-treated livers, but increased even more in livers treated with polyIC or the combination (FIG. 5C).

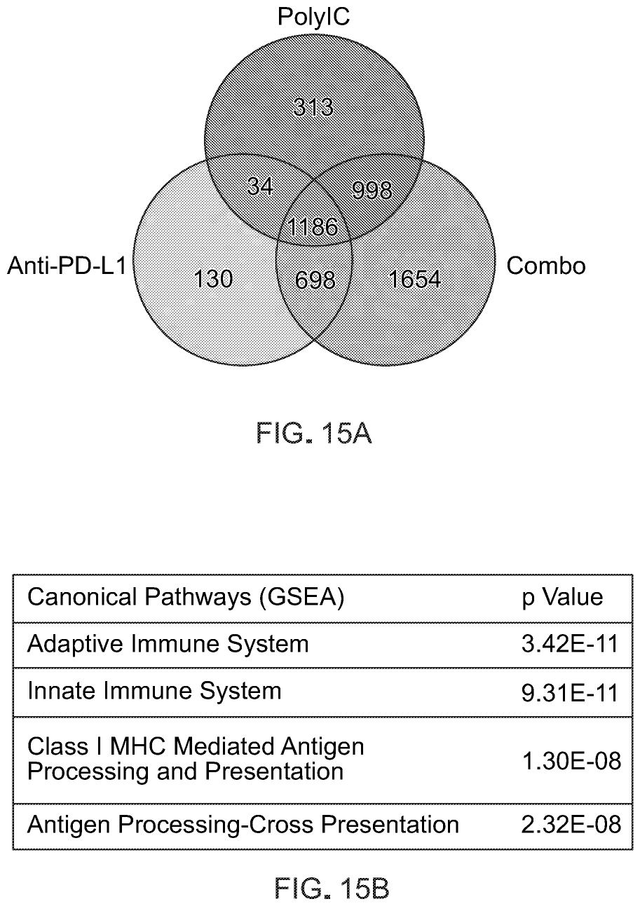

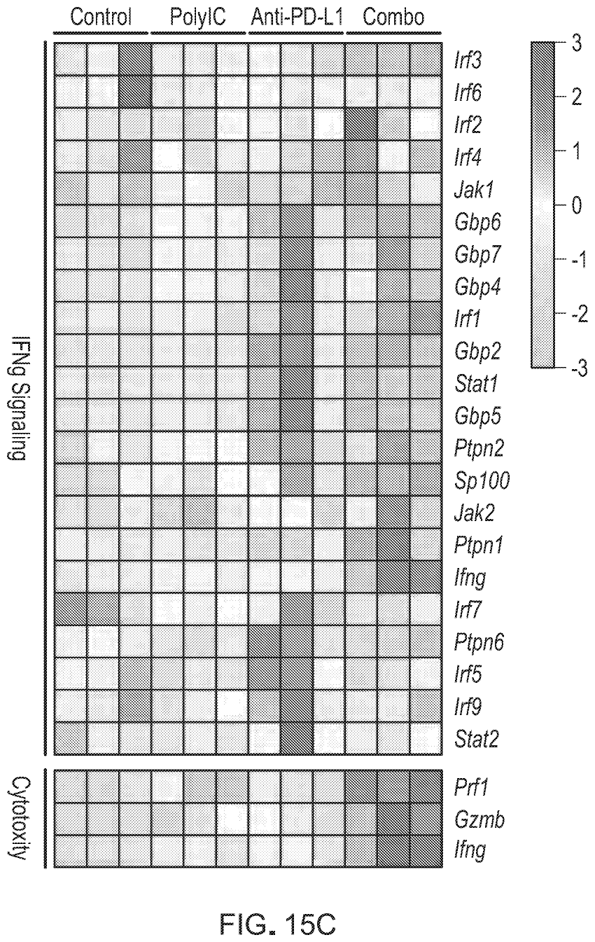

To further investigate the underlying mechanism, RNA-seq analysis of isolated CD45+ immune cells infiltrated into the liver was performed. Using cuffdiff, data was acquired and analyzed on gene expression levels in different groups. The differentially expressed genes, including up- and down-regulated, were identified by cut-off of 2-fold with q-value of <0.05. The list in FIG. 15A showed a total of 1654 differentially expressed genes specifically in the combination group. Gene set enrichment analysis (GSEA) of these 1654 genes demonstrated that the immune cells in the combination group were particularly associated with adaptive and innate immune system and the progresses of antigen processing and presentation, pointing to an enhanced anti-tumor immunity (FIG. 15B). The key genes involved in interferon-γ (IFNγ) signaling pathway were also listed (FIG. 15C, top), which was regarded as sign of activated anti-tumor immunity. A heat map showed that the combination treatment maximized IFNγ signaling in hepatic immune cells. Also upregulated most profoundly in the combination group were some key genes, perforin (Prf1), granzyme B (Gzmb), IFNγ (Ifng), indicators of anti-tumor cytotoxic function (FIG. 15C, bottom). Therefore, RNA-seq data further confirmed that combination of polyIC and PD-L1 Ab dramatically enhanced the anti-tumor immunity in the liver, especially the adaptive immune response.

### The Effect of Combination Therapy is Cytotoxic T Cell-Dependent

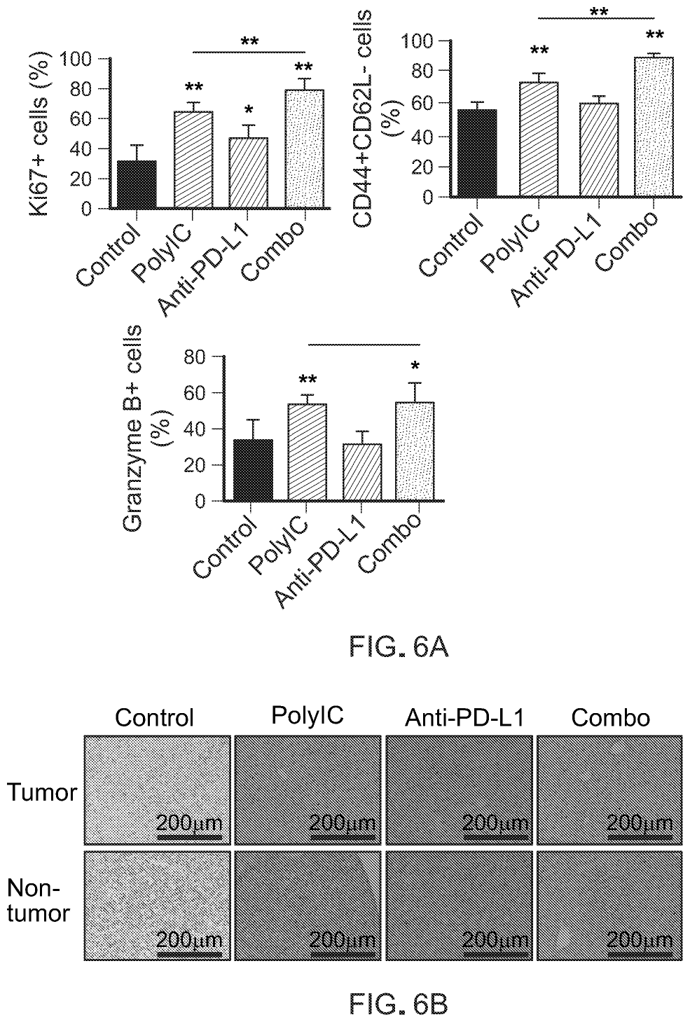

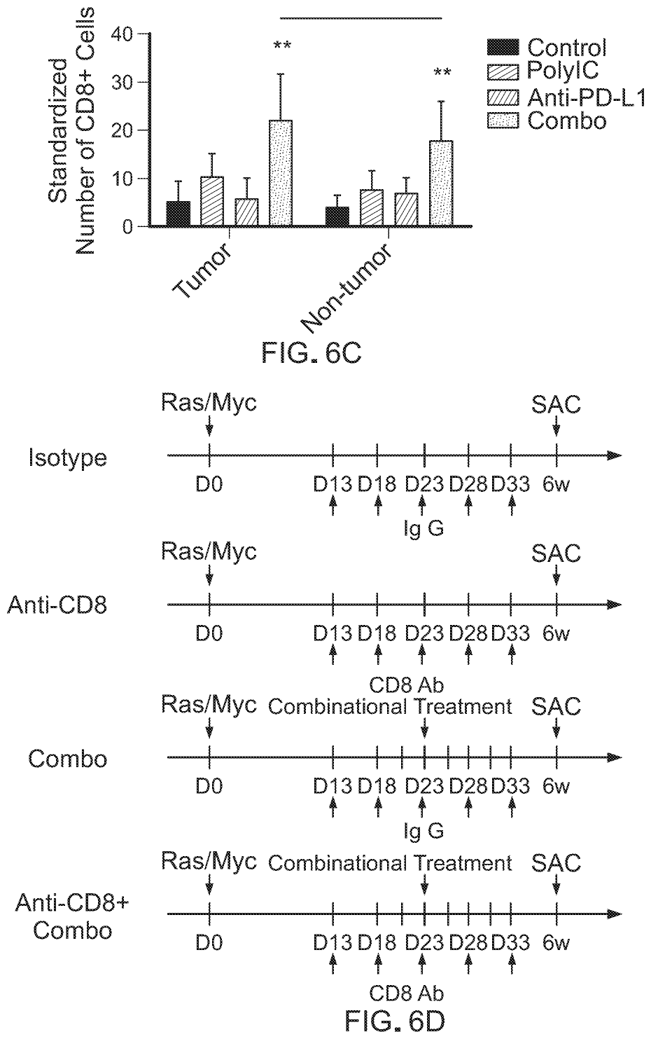

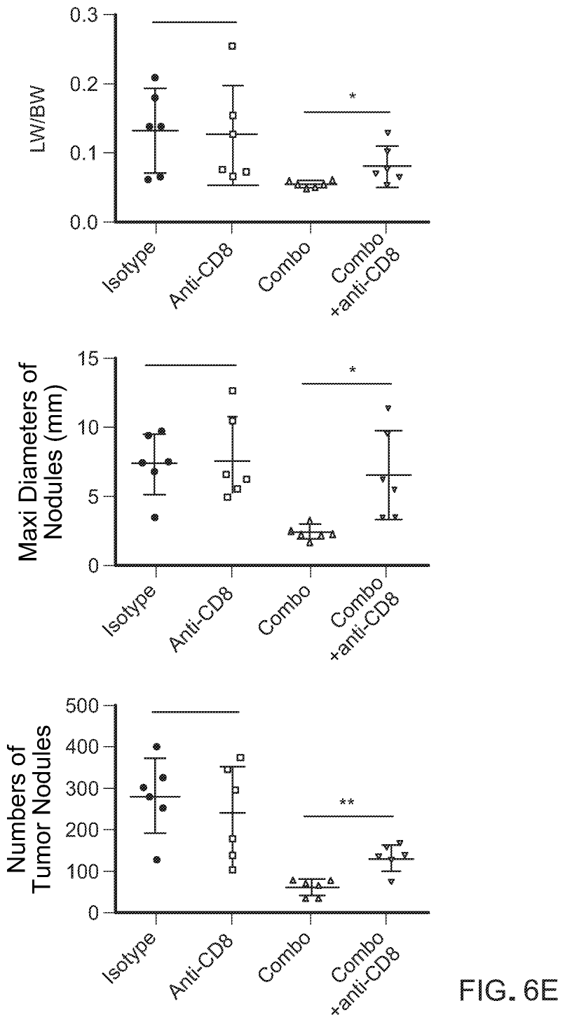

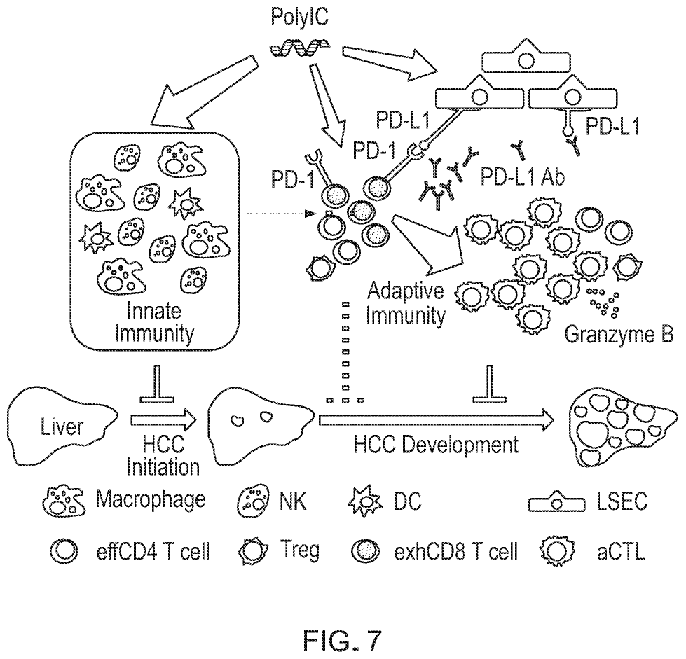

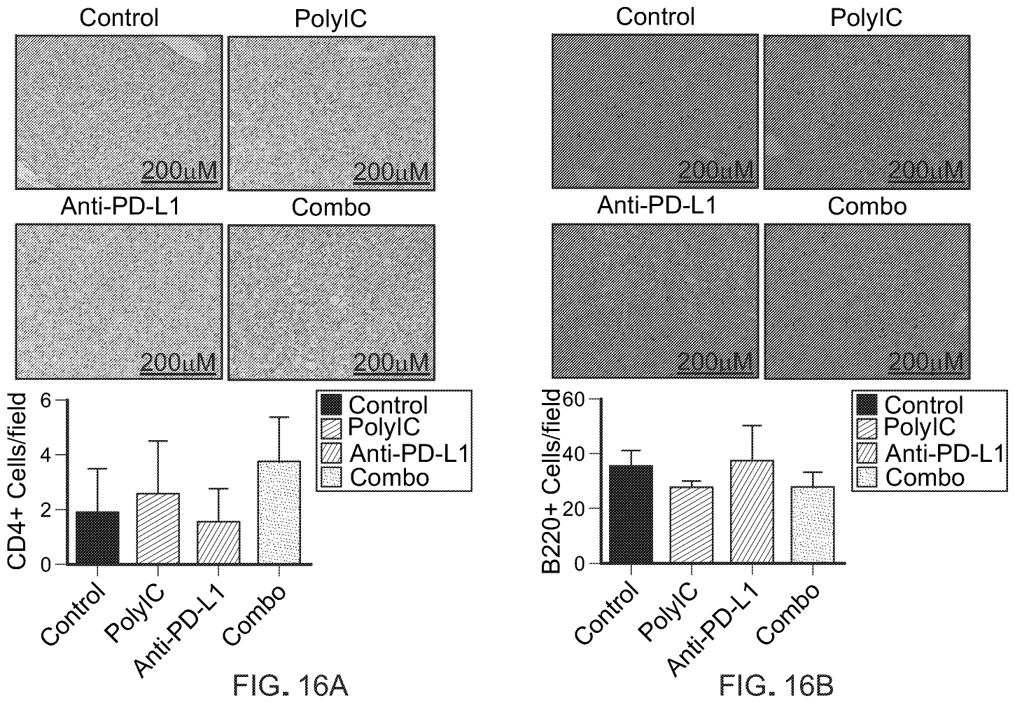

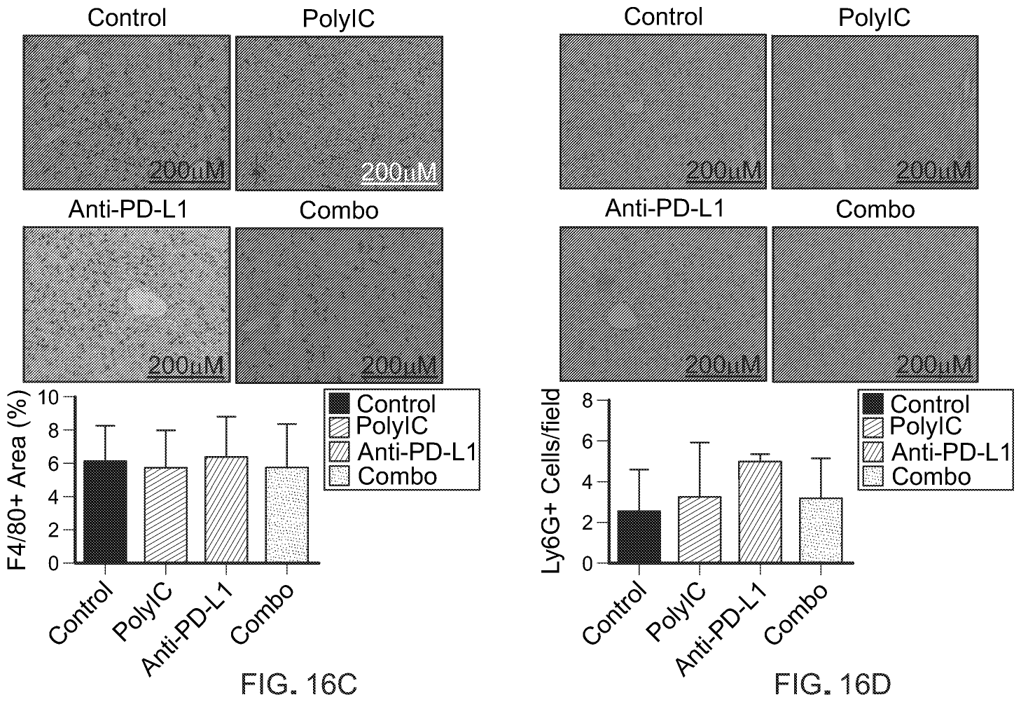

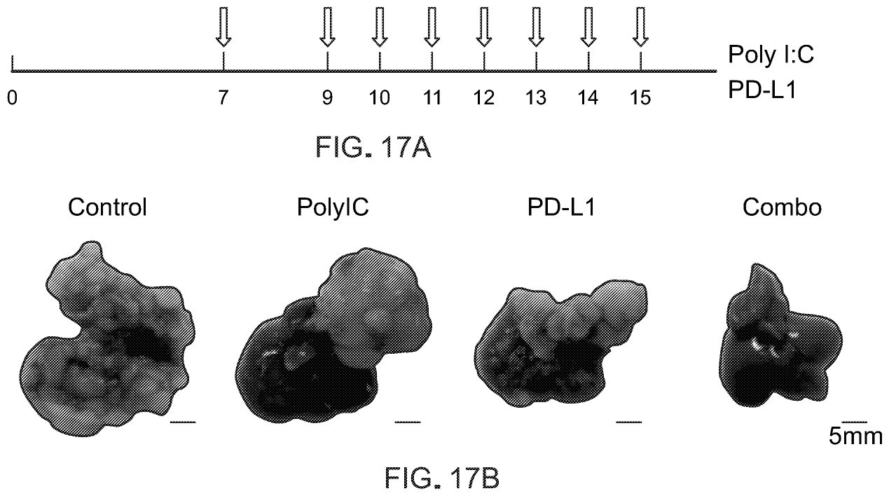

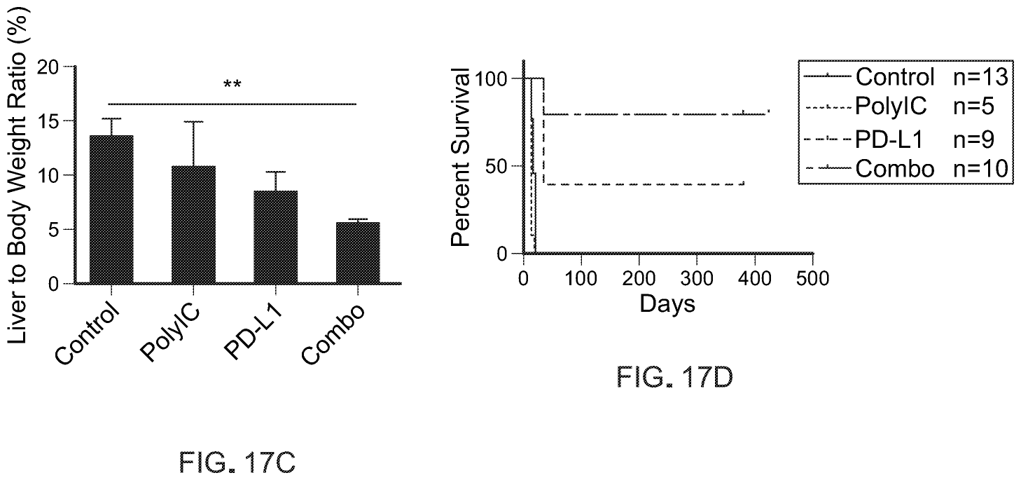

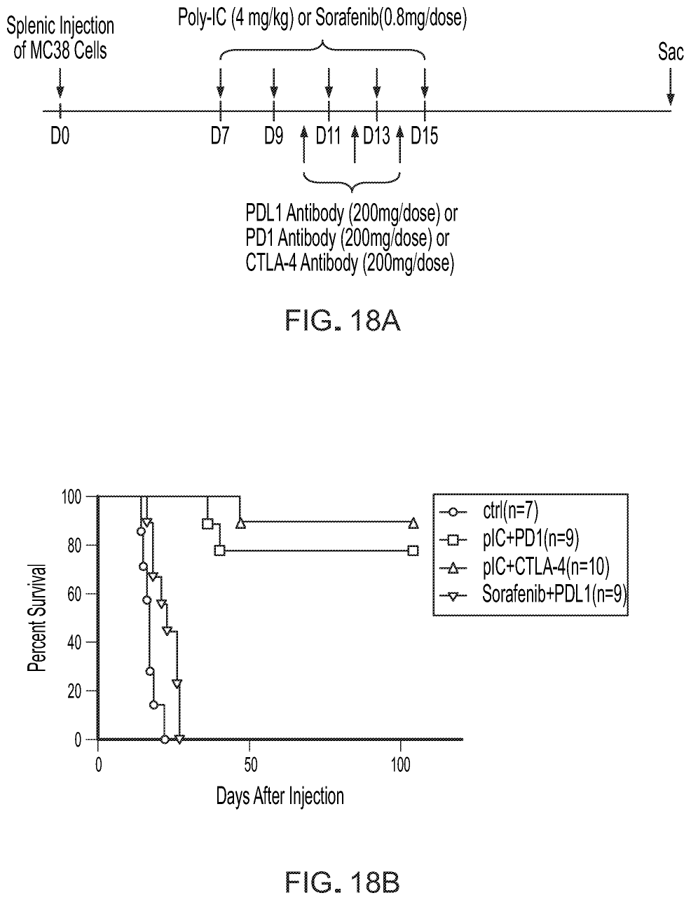

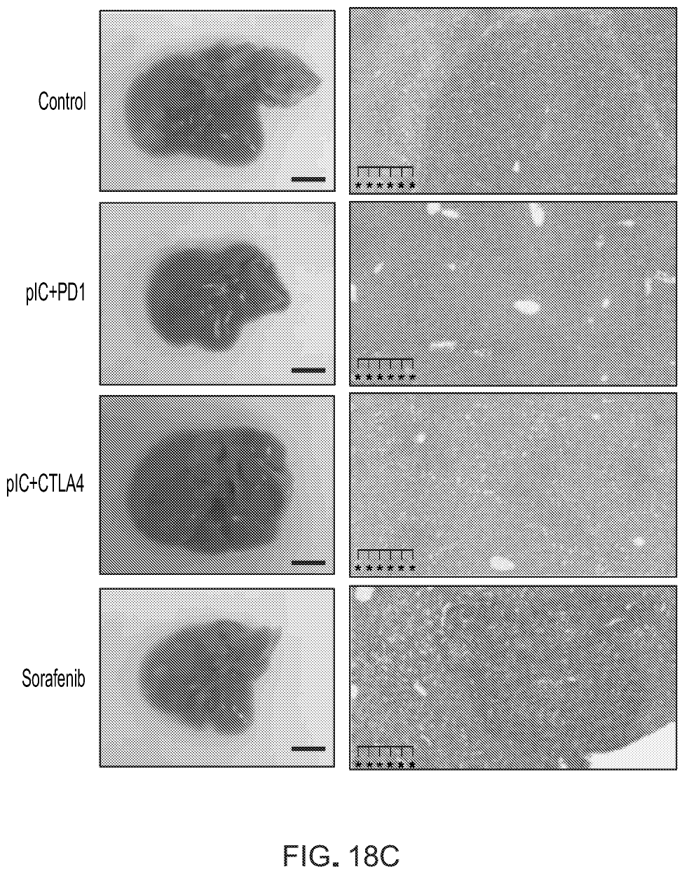

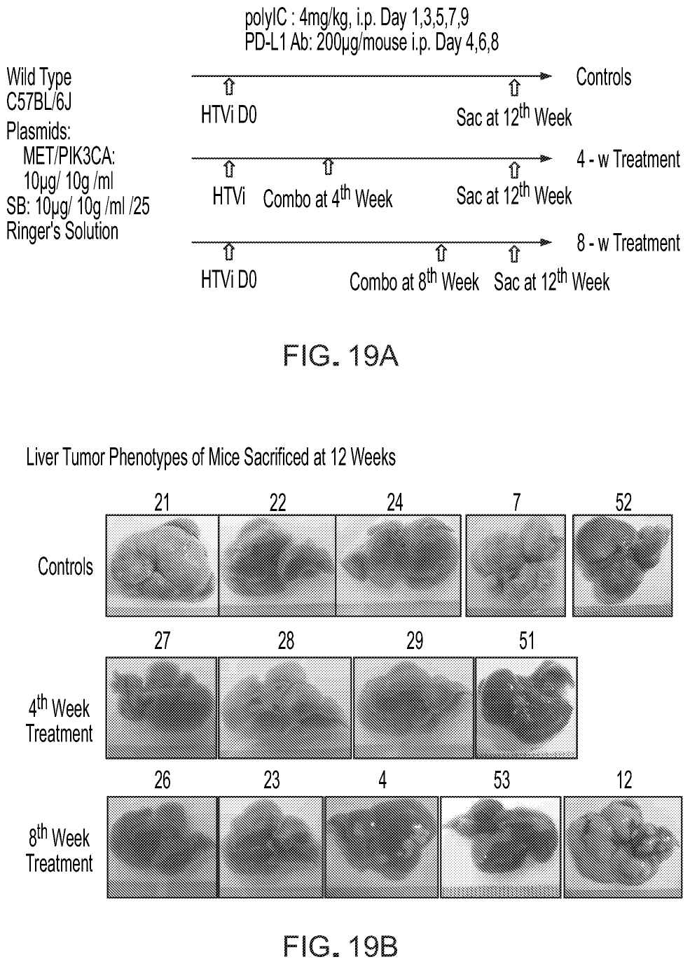

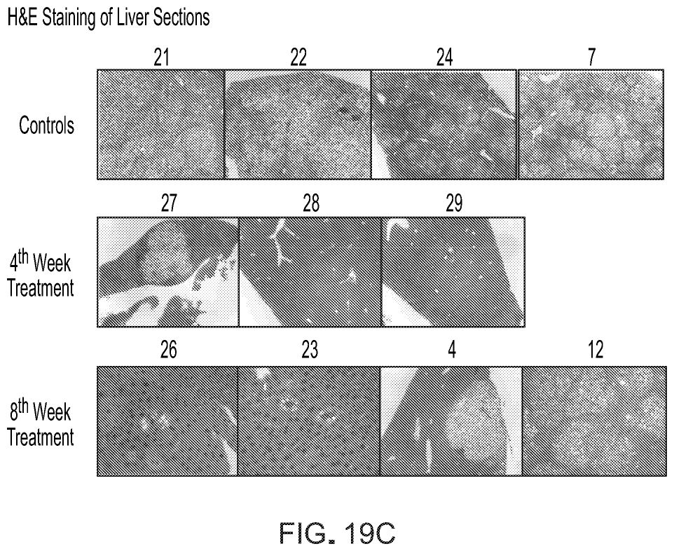

Evidently, the combination of polyIC and PD-L1 Ab caused dramatic accumulation of CD8 T cells and activation of their cytotoxic activities. Changes were further examined of CD8 T cells immediately after various treatments as shown in FIG. 5A. Flow cytometry was performed to analyze the proliferation (Ki67+), activation (CD44+CD62L−) and cytotoxic function (Granzyme B+) of CD8 T cells (FIG. 14C). Interestingly, PD-L1 Ab treatment only slightly enhanced the activation of CD8 T cells but failed to affect its proliferation and cytotoxic function. polyIC treatment enhanced proliferation, activation and cytotoxic function of CD8 T cells, compared to the control, and its combination with PD-L1 Ab further boosted proliferation and activation of CD8 T cells (FIG. 6A). Therefore, the combination of PD-L1 Ab with polyIC rapidly induced drastic accumulation, proliferation and activation of CD8 cytotoxic T cells, resulting in tumor suppression in the liver. It was then asked if these changes of CD8 T cells detected in the early phase were maintained in the late phase. Immunostaining of liver sections collected at 6 weeks (as shown in FIG. 4A) showed that the numbers of CD8 T cells in both tumor and non-tumor areas were much higher in the combination group than other groups (FIG. 6B-C). However, the accumulation of CD4 T cells, B cells, macrophages and neutrophils was similar among the four groups at the late stage (FIG. 16A-D). Thus, the combination treatment induced a sustained CD8 T cell accumulation in the liver. Finally, to determine a functional requirement of CD8 T cells in the combination therapy, CD8 Ab to block CD8 T cell function were injected (FIG. 6D). Although CD8 Ab did not affect HCC development in this model, CD8 blockade impaired the tumor-inhibitory effect of the combined treatment (FIG. 6E), indicating a critical role of cytotoxic CD8 T cells in mediating the anti-tumor immunity.

### DISCUSSION

Synthetic dsRNA polyIC has a tumor-preventative effect in several mouse models for liver tumors induced by transfection of oncogenes and chemical carcinogen DEN, or even spontaneously developed due to Pten deficiency and associated non-alcoholic steatohepatitis (NASH). Notably, polyIC injection enhances accumulation and activation of innate immune cells in the liver, especially NK cells and macrophages, independent of tumor development. Coordinated activation of these innate immune cells leads to elimination of transforming hepatocytes or TICs, resulting in prevention of tumor initiation.

As a putative cancer vaccine adjuvant, polyIC was shown to induce secretion of various pro-inflammatory cytokines by different immune cell types and tumor cells. However, very minor effects of polyIC on DC in the liver were detected, but polyIC induced reprogramming of macrophage polarization towards M1 phenotype and NK cell activation in the liver. It is likely that coordinated activation of multiple innate immunity by polyIC caused cell senescence and clearance of TICs in DEN-treated livers and Pten-deficient fatty livers. Consistently, polyIC was shown to activate NK cells and upregulate their cytolytic activity. The current study confirmed accumulation of NK cells and macrophages in polyIC-treated livers, and also demonstrated the requirement of NK cells and macrophages, as depletion of the two cell subsets abrogated the inhibitory effect of polyIC on liver tumorigenesis. These results suggest that boosting innate immunity by pharmaceuticals or other means may be a powerful strategy to prevent tumorigenesis in a huge group of subjects with chronic liver diseases who are at high risk for HCC.

Despite a tumor-preventive role of polyIC given at the pre-caner stage, it was consistently shown that administration of polyIC during tumor progression did not have significant therapeutic effect. Further, it was found that injection of polyIC alone may even aggravate liver tumorigenesis that is highly associated with inflammation.

The mechanisms of why polyIC was insufficient to suppress tumor progression, when injected after tumor initiation was analyzed. In addition to its activation of innate immunity, polyIC also modulated the adaptive immune functions, with modest accumulation of CD8 T cells in the liver. In spite of the decreased accumulation of MDSC, polyIC also elevated the ratio of Treg cells. Although CD8 T cell accumulation was enhanced, the dsRNA induced remarkably elevated PD-1 expression in CD4 and CD8 T cells. Together, these multiple factors likely compromised its anti-tumor effect if administered during tumor progression. The elevated expression of PD-1 in T cells prompted an interrogation of the underlying mechanism. Consistent with the previous RNA-seq analysis detecting increased CD274 (PD-L1) expression in polyIC-treated liver immunoblot analysis showed remarkable increase of PD-L1 in livers treated with polyIC, regardless of the background (FIG. 3A). These results argue that while activating multiple innate immune activities, polyIC also promotes signaling through the PD-L1/PD-1 axis in the liver. Thus, treatment with polyIC alone may cause T cell anergy or exhaustion, dampening the anti-tumor immunity.

However, this data also inspired to test an idea of combined treatment of polyIC and PD-L1 blockade. Indeed, the data in this example indicate high efficacy of this combination therapy. Potent suppression of tumor progression was observed in several mouse models driven by oncogenes frequently detected in human HCC patients, and the effect was observed when injecting the two reagents at early or advanced stages of tumor progression. Mechanistically, the combination most efficiently boosted hepatic infiltration of activated CD8 T cells, and anti-CD8 antibody attenuated the tumor-suppressing effect. Of note, polyIC induced PD-L1 expression in LSECs, but not in tumor cells or other NPCs, in the liver. LSECs constitute a special structural component and function as the first immune barrier against gut microbiota and a platform for nutrient exchange through endocytosis. More recently, LSEC has been recognized as a special type of antigen-presenting cell (APC) in the liver, capable of cross-presenting soluble exogenous antigens to CD8 T cells, leading to its tolerance.⁽³⁷⁾ It was also shown that circulating CEA was preferentially taken up and cross-presented by LSECs, but not dendritic cells, to promote immune tolerance of CD8 T cells through B7H1 (PD-L1) expression.⁽³⁸⁾ PolyIC-triggered activation of PD-L1/PD-1 signaling may be harnessed to boost anti-tumor immunity in the liver via sustained accumulation of activated cytotoxic CD8 T cells by concurrent PD-L1 blockade.

In experiments with combined treatment of polyIC and PD-L1 blockade, injection of PD-L1 Ab alone failed to see any tumor-inhibitory effect in the mouse HCC models. Consistently, an obvious PD-L1 upregulation during HCC progression was not observed, in tumor cells, surrounding hepatocytes or NPCs, which predicts low anti-tumor response of PD-L1 blockade in liver cancer. Encouraged by beneficial responses in some patients with solid tumors, checkpoint inhibitors are already in numerous clinical trials for HCC patients worldwide, although the outcomes are uncertain. One serious concern is the low or no response of HCC due to the unusual immunosuppressive liver environment. This issue may be remedied by concerted activation of both innate and acquired immune functions in the liver. As polyIC induction of PD-L1 in LSECs was independent of tumor formation, this therapeutic strategy can be applied to HCCs of various etiologies. The combined treatment showed therapeutic efficacy at early and late stages of tumor progression, with complete tumor remission and tumor-free survival observed in a few mice.

### OTHER EMBODIMENTS

It is to be understood that while the invention has been described in conjunction with the detailed description thereof, the forgoing description is intended to illustrate and not limit the scope of the invention, which is defined by the scope of the appended claims. Other aspects, advantages, and modifications are within the scope of the following claims.

### REFERENCES

1. Ferlay J, Soerjomataram I, Dikshit R, Eser S, Mathers C, Rebelo M, Parkin D M, et al. Cancer incidence and mortality worldwide: sources, methods and major patterns in GLOBOCAN 2012. Int J Cancer 2015; 136:E359-386.
2. Llovet J M, Zucman-Rossi J, Pikarsky E, Sangro B, Schwartz M, Sherman M, Gores G. Hepatocellular carcinoma. Nat Rev Dis Primers 2016; 2:16018.
3. Ryerson A B, Eheman C R, Altekruse S F, Ward J W, Jemal A, Sherman R L, Henley S J, et al. Annual Report to the Nation on the Status of Cancer, 1975-2012, featuring the increasing incidence of liver cancer. Cancer 2016; 122:1312-1337.
4. Llovet J M, Ricci S, Mazzaferro V, Hilgard P, Gane E, Blanc J F, de Oliveira A C, et al. Sorafenib in advanced hepatocellular carcinoma. N Engl J Med 2008; 359:378-390.
5. European Association For The Study Of The L, European Organisation For R, Treatment Of C. EASL-EORTC clinical practice guidelines: management of hepatocellular carcinoma. J Hepatol 2012; 56:908-943.
6. Bruix J, Qin S, Merle P, Granito A, Huang Y H, Bodoky G, Pracht M, et al. Regorafenib for patients with hepatocellular carcinoma who progressed on sorafenib treatment (RESORCE): a randomised, double-blind, placebo-controlled, phase 3 trial. Lancet 2017; 389:56-66.
7. Kudo M, Finn R S, Qin S, Han K H, Ikeda K, Piscaglia F, Baron A, et al. Lenvatinib versus sorafenib in first-line treatment of patients with unresectable hepatocellular carcinoma: a randomised phase 3 non-inferiority trial. Lancet 2018; 391:1163-1173.
8. Abou-Alfa G K, Meyer T, Cheng A L, El-Khoueiry A B, Rimassa L, Ryoo B Y, Cicin I, et al. Cabozantinib in Patients with Advanced and Progressing Hepatocellular Carcinoma. N Engl J Med 2018; 379:54-63.
9. Llovet J M, Hernandez-Gea V. Hepatocellular carcinoma: reasons for phase III failure and novel perspectives on trial design. Clin Cancer Res 2014; 20:2072-2079.
10. Chen L, Han X. Anti-PD-1/P D-L1 therapy of human cancer: past, present, and future. J Clin Invest 2015; 125:3384-3391.
11. Whiteside T L, Demaria S, Rodriguez-Ruiz M E, Zarour H M, Melero I. Emerging Opportunities and Challenges in Cancer Immunotherapy. Clin Cancer Res 2016; 22:1845-1855.
12. Topalian S L, Drake C G, Pardoll D M. Immune checkpoint blockade: a common denominator approach to cancer therapy. Cancer Cell 2015; 27:450-461.
13. Makarova-Rusher O V, Medina-Echeverz J, Duffy A G, Greten T F. The yin and yang of evasion and immune activation in HCC. J Hepatol 2015; 62:1420-1429.
14. Hato T, Goyal L, Greten T F, Duda D G, Zhu A X. Immune checkpoint blockade in hepatocellular carcinoma: current progress and future directions. Hepatology 2014; 60:1776-1782.
15. El-Khoueiry A B, Sangro B, Yau T, Crocenzi T S, Kudo M, Hsu C, Kim T Y, et al. Nivolumab in patients with advanced hepatocellular carcinoma (CheckMate 040): an open-label, non-comparative, phase 1/2 dose escalation and expansion trial. Lancet 2017; 389:2492-2502.
16. Zhu A X, Finn R S, Edeline J, Cattan S, Ogasawara S, Palmer D, Verslype C, et al. Pembrolizumab in patients with advanced hepatocellular carcinoma previously treated with sorafenib (KEYNOTE-224): a non-randomised, open-label phase 2 trial.

Lancet Oncol 2018; 19:940-952.
17. Jenne C N, Kubes P. Immune surveillance by the liver. Nat Immunol 2013; 14:996-1006.
18. Gao B, Jeong W I, Tian Z. Liver: An organ with predominant innate immunity. Hepatology 2008; 47:729-736.
19. Greten T F, Sangro B. Targets for immunotherapy of liver cancer. J Hepatol 2017.
20. Lee J, Liao R, Wang G, Yang B H, Luo X, Varki N M, Qiu S J, et al. Preventive Inhibition of Liver Tumorigenesis by Systemic Activation of Innate Immune Functions. Cell Rep 2017; 21:1870-1882.
21. Chen X, Calvisi D F. Hydrodynamic transfection for generation of novel mouse models for liver cancer research. The American journal of pathology 2014; 184:912-923.
22. Liu J J, Li Y, Chen W S, Liang Y, Wang G, Zong M, Kaneko K, et al. Shp2 deletion in hepatocytes suppresses hepatocarcinogenesis driven by oncogenic beta-Catenin, PIK3C A and MET. J Hepatol 2018; 69:79-88.
23. Liang Y, Feng Y, Zong M, Wei X F, Lee J, Feng Y, Li H, et al. beta-catenin deficiency in hepatocytes aggravates hepatocarcinogenesis driven by oncogenic beta-catenin and MET. Hepatology 2018; 67:1807-1822.
24. Heinecke L, Proud D, Sanders S, Schleimer R P, Kim J. Induction of B7-H1 and B7-DC expression on airway epithelial cells by the Toll-like receptor 3 agonist double-stranded RNA and human rhinovirus infection: In vivo and in vitro studies. J Allergy Clin Immunol 2008; 121:1155-1160.
25. Telcian A G, Laza-Stanca V, Edwards M R, Harker J A, Wang H, Bartlett N W, Mallia P, et al. RSV-induced bronchial epithelial cell PD-L1 expression inhibits CD8+ T cell nonspecific antiviral activity. J Infect Dis 2011; 203:85-94.
26. Salmon H, Idoyaga J, Rahman A, Leboeuf M, Remark R, Jordan S, Casanova-Acebes M, et al. Expansion and Activation of CD103(+) Dendritic Cell Progenitors at the Tumor Site Enhances Tumor Responses to Therapeutic PD-L1 and BRAF Inhibition. Immunity 2016; 44:924-938.
27. Hong S, Chen N, Fang W, Zhan J, Liu Q, Kang S, He X, et al. Upregulation of PD-L1 by EML4-ALK fusion protein mediates the immune escape in ALK positive NSCLC: Implication for optional anti-PD-1/P D-L1 immune therapy for ALK-TKIs sensitive and resistant NSCLC patients. Oncoimmunology 2016; 5:e1094598.
28. Shang N, Arteaga M, Zaidi A, Stauffer J, Cotler S J, Zeleznik-Le N J, Zhang J, et al. FAK is required for c-Met/beta-catenin-driven hepatocarcinogenesis. Hepatology 2015; 61:214-226.
29. Zucman-Rossi J, Villanueva A, Nault J C, Llovet J M. Genetic Landscape and Biomarkers of Hepatocellular Carcinoma. Gastroenterology 2015; 149:1226-1239 e1224.
30. Dunn G P, Koebel C M, Schreiber R D. Interferons, immunity and cancer immunoediting. Nat Rev Immunol 2006; 6:836-848.
31. Ammi R, De Waele J, Willemen Y, Van Brussel I, Schrijvers D M, Lion E, Smits E L. Poly(I:C) as cancer vaccine adjuvant: knocking on the door of medical breakthroughs. Pharmacol Ther 2015; 146:120-131.
32. Datta S K, Redecke V, Prilliman K R, Takabayashi K, Corr M, Tallant T, DiDonato J, et al. A subset of Toll-like receptor ligands induces cross-presentation by bone marrow-derived dendritic cells. J Immunol 2003; 170:4102-4110.
33. Schulz O, Diebold S S, Chen M, Naslund T I, Nolte M A, Alexopoulou L, Azuma Y T, et al. Toll-like receptor 3 promotes cross-priming to virus-infected cells. Nature 2005; 433:887-892.
34. McCartney S, Vermi W, Gilfillan S, Cella M, Murphy T L, Schreiber R D, Murphy K M, et al. Distinct and complementary functions of MDA5 and TLR3 in poly(I:C)-mediated activation of mouse N K cells. J Exp Med 2009; 206:2967-2976.
35. Sivori S, Falco M, Della Chiesa M, Carlomagno S, Vitale M, Moretta L, Moretta A. CpG and double-stranded RNA trigger human N K cells by Toll-like receptors: induction of cytokine release and cytotoxicity against tumors and dendritic cells. Proc Natl Acad Sci USA2004; 101:10116-10121.
36. Cheever M A. Twelve immunotherapy drugs that could cure cancers. Immunol Rev 2008; 222:357-368.
37. Limmer A, Ohl J, Kurts C, Ljunggren H G, Reiss Y, Groettrup M, Momburg F, et al. Efficient presentation of exogenous antigen by liver endothelial cells to CD8+ T cells results in antigen-specific T-cell tolerance. Nat Med 2000; 6:1348-1354.
38. Hochst B, Schildberg F A, Bottcher J, Metzger C, Huss S, Turler A, Overhaus M, et al. Liver sinusoidal endothelial cells contribute to CD8 T cell tolerance toward circulating carcinoembryonic antigen in mice. Hepatology 2012; 56:1924-1933.

1. A method of treating a cancer in a subject in need thereof, the method comprising administering a therapeutically effective amount of a double stranded RNA (dsRNA).

2. The method of claim 1, wherein the method comprises administering a therapeutically effective amount of a double stranded RNA (dsRNA) and an antibody.

3. The method of claim 1, wherein the dsRNA is a TLR3 ligand.

4. The method of claim 1, wherein the dsRNA is a TLR3 agonist.

5. The method of claim 1, wherein the dsRNA is selected from the group consisting of a polyadenylic-polyuridylic acid or a polyinosinic-polycytidylic acid.

6. The method of claim 1, wherein the polyinosinic-polycytidylic acid is polyIC.

7-16. (canceled)

17. The method of claim 2, wherein the antibody is an anti-PD-L1 antibody.

18. The method of claim 1, wherein the cancer is a liver cancer.

19. The method of claim 1, wherein the cancer is primary liver cancer.

20. The method of claim 1, wherein the cancer is late stage liver cancer.

21. The method of claim 1, wherein the cancer is metastatic colon cancer with liver tumors.

22. The method of claim 1, wherein the cancer is hepatocellular carcinoma (HCC).

23. The method of claim 1, wherein the cancer expresses an oncogene selected from the group consisting of N-Ras, c-Myc, c-Met, or a truncated beta-catenin mutant.

24-34. (canceled)

35. A pharmaceutical combination comprising a dsRNA and an antibody.

36. The pharmaceutical combination of claim 35, wherein the dsRNA is polyIC.

37. The pharmaceutical combination of claim 35, wherein the antibody is anti-PD-L1.

38. The pharmaceutical combination of claim 35, wherein the dsRNA and antibody are administered as a fixed combination.

39. The pharmaceutical combination of claim 35, wherein the dsRNA and antibody are administered as a non-fixed combination.

40. The pharmaceutical combination of claim 35, wherein the dsRNA and antibody are administered sequentially.

41. The pharmaceutical combination of claim 35, wherein the dsRNA and antibody are administered concurrently.


SEQUENCE LISTING (6 sequences)

SEQ ID NO: 1 - DNA, 20 bp, Mus musculus
ccgaccacatgaagcagcac

SEQ ID NO: 2 - DNA, 20 bp, Mus musculus
tcgccctcgaacttcacctc

SEQ ID NO: 3 - DNA, 22 bp, Mus musculus
attgctccttgactgctggctg

SEQ ID NO: 4 - DNA, 22 bp, Mus musculus
attgctccttgactgctggctg

SEQ ID NO: 5 - DNA, 28 bp, Mus musculus
cgacttcaacagcaactcccactcttcc

SEQ ID NO: 6 - DNA, 27 bp, Mus musculus
tgggtggtccagggtttcttactcctt